(19) United States
(12) Patent Application Publication
(10) Pub. No.: US 2004/0142387 A1
(43) Pub. Date: Jul. 22, 2004

(54) Mutants of GAD65 and Ian5 relating to diabetes

(75) Inventors: Ake Lernmark, Seattle, WA (US); Dong Luo, Kenmore, WA (US); Armand MacMurray, Seattle, WA (US); Ruth A. Ettinger, Seattle, WA (US); Daniel Moralejo, Seattle, WA (US); Elizabeth A. Rutledge, Lake Stevens, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: University of Washington, Seattle, WA (US)

(21) Appl. No.: 10/449,741

(22) Filed: May 29, 2003

Related U.S. Application Data
(60) Provisional application No. 60/383,913, filed on May 29, 2002.

(51) Int. Cl. G01N 33/53; G01N 33/567; C07K 14/47

(52) U.S. Cl. 435/7.2; 530/350

(57) ABSTRACT

GAD65 mutants which lack a C-terminal conformational epitope are disclosed. The GAD65 mutants can be used in diagnostic methods for detecting the presence of or risk of developing type 1 diabetes. Also disclosed are compositions relating to the Ian5 gene, including Ian5 polynucleotides, Ian5 polypeptides and antibodies thereto, and expression vectors, recombinant cells comprising the Ian5 polynucleotides, and genetically modified animal models. In particular, the compositions include those relating to truncated mutant Ian5 polypeptides lacking a significant portion of the C-terminus. Mutations in the Ian5 gene locus that result in a truncated Ian5 protein are associated with lymphopenia and type 1 diabetes in mammals. The compositions are useful, for example, in methods of screening for agonists or antagonists of Ian5 pathways, such as to identify candidate agents for diabetes drug development, or for developing gene therapy for type 1 diabetes or a related disorder. Also disclosed are diagnostic methods relating to Ian5 gene mutations.

### CROSS-REFERENCES TO RELATED APPLICATIONS

This application claims priority to U.S. provisional application No. 60/383,913, filed on May 29, 2002, incorporated by reference herein in its entirety for all purposes.

### STATEMENT AS TO RIGHTS TO INVENTIONS MADE UNDER FEDERALLY SPONSORED RESEARCH OR DEVELOPMENT

This work was supported in part by grants A142380 and DK26190-14 from the National Institutes of Health. The U.S. Governnent may have certain rights in the invention.

### BACKGROUND OF THE INVENTION

Type 1 (insulin dependent) diabetes mellitus in humans is a significant health problem with a prevalence ranging from 0.3-1% in different populations (Onkamo et al., Diabetologia 42:1395-1403 (1999)). Type 1 diabetes mellitus develops after selective destruction of pancreatic islet β cells in association with several autoimmune phenomena. Although multiple autoantigens have been implicated, the 65 kDa isoform (GAD65) of glutamic acid decarboxylase (Karlsen et al., Proc. Natl. Acad. Sci. USA 88:8337-8341 (1991), Karlsen et al., Diabetes 41:1355-1359 (1992)), unlike GAD67, is recognized as a major self-antigen (Schranz et al., Diab. Metab. Rev. 14:3-29 (1998)). The two GAD isoforms are highly homologous and differ mostly in the amino terminal-third of the protein (Bu et al., Proc. Natl. Acad. Sci. USA 89:2115-2119 (1992)).

Because of the high diagnostic sensitivity for type 1 diabetes, the presence of GAD65 autoantibodies (GAD65Ab) are currently used to identify subjects at risk (Verge et al., Diabetes 45:926-933 (1996)). GAD65Ab represent early markers of β-cell autoimmunity and the diagnostic sensitivity is as high as 80% (Baekkeskov et al., Nature 347:151-156 (1990)). However, the occurrence of this marker in about 1% of healthy individuals (Rolandsson et al., Diabetologia 42:555-5559 (1999)) and in patients with other autoimmune diseases (Lernmark, J. Internal Med. 240:259-277 (1996)), including the rare neurological disorder, stiff-man syndrome (SMS) (Kim et al., J. Exp. Med. 180:595-606 (1994), Solimena et al., N. Engl. J. Med. 322:1555-1560 (1990)) which is not necessarily associated with type 1 diabetes, limits its diagnostic specificity.

Studies have also demonstrated many genetic factors contributing to type 1 diabetes, including both major histocompatibility complex (MHC) and non-MHC loci (See, e.g., Graham et al., Diabetes 51:1346-1355 (2002); Nerup et al., Lancet 2:864-866 (1974); Todd et al., Nature 329:599-604 (1987)). For example, non-MHC loci contributing to type 1 diabetes have been identified in the diabetes prone BB rat (BBDP), one of the best models of spontaneous autoimmune diabetes. (Jacob et al., Nature Genetics 2:56-60 (1992); Klaff et al., Mamm. Genome 10:883-887 (1999); Kwitek et al., FASEB 16:A864 (2002).) The Iddml/lyp locus, linked to peripheral T cell lymphopenia (<15% normal T cell count) and type 1 diabetes, has been mapped to a 0.7 cM interval on rat chromosome 4 (Jacob et al., supra; Homum et al., Mamm. Genome 6:371-372 (1995); Klaff et al., supra; Kloting et al., Acta Diabetol 35:109-111 (1998)). In the BBDP diabetes model, lymphopenia is essential for the development of the diabetic phenotype and is inherited as a simple Mendelian trait (Jacob et al., supra; Bieg et al., Mamm. Genome 9:324-326 (1998)). Lymphopenia has also been observed in human type I diabetes patients with a family history of the disease, as well as in their first degree relatives (See, e.g., Kaaba and Al-Harbi, Immunol. Lett. 47:209-13 (1995).)

Studies of the Iddml/lyp locus in the absence of the other Iddm loci in congenic rat strains have confirmed that a single locus is responsible for both T cell lymphopenia and spontaneous autoimmune diabetes. (Bieg et al., Mamm. Genome 9:324-326 (1998).) In the completed congenic DR.lyp line, and in recombinant animals developed from this strain, no animal developed diabetes without lymphopenia (lyp) (Id.), suggesting that the lymphocytopenia gene is responsible for the loss of critical T cells, resulting in autoimmunity. Specific pathogen free wild type (+/+) and heterozygous (lyp/+) DR.lyp rats have normal lymphocyte numbers and do not develop diabetes, while DR.lyp/lyp rats have T cell lymphopenia from birth and clinical onset of type 1 diabetes between 50 and 108 days of age in 100% of the animals (Bieg et al., supra; Klaff et al, supra). Iddml/lyp gene function is therefore critical to the development of age-dependent type 1 diabetes.

### BRIEF SUMMARY OF THE INVENTION

In one aspect, the present invention provides mutant GAD65 polypeptides that comprise an E517P mutation and that is characterized by decreased specific binding to GAD6, MICA-1, MICA-3, MICA-4, or MICA-6 antibody. The decreased binding is relative when compared to a corresponding GAD65 polypeptide not having the E517P mutation. In certain embodiments, the mutant GAD65 polypeptide is also characterized by decreased specific binding to MICA-2 antibody, with the decreased binding again relative to a corresponding GAD65 polypeptide not having the E517P mutation. In other embodiments, the mutant GAD65 polypeptide is a full-length GAD65 polypeptide. In yet other embodiments, the mutant GAD65 polypeptide can include a sequence at position 515-525 that is either SEQ ID NO: 15 or SEQ ID NO: 16.

In another aspect, the present invention provides a method for detecting the presence or risk of type 1 diabetes in a subject. The method comprises the following steps: (1) isolating from a subject a first and second serum sample; (2) contacting the first serum sample with a mutant GAD65 polypeptide that comprises an E517P mutation and that is characterized by decreased specific binding to GAD6, MICA-1, MICA-3, MICA-4, or MICA-6 antibody, where the decreased binding is relative when compared to a corresponding GAD65 polypeptide not having the E517P mutation; (3) contacting the second serum sample with a control GAD65 polypeptide that is immunologically cross-reactive with C-terminal conformational epitopes of wild-type GAD65, said control GAD65 polypeptide not having the E517P mutation and having substantially the same GAD6-, MICA-1-, MICA-3-, MICA-4-, or MICA-6-specific binding activity as the corresponding GAD65 polypeptide; (4) determining the degree of GAD65 autoantibody binding activity in the first and second serum samples; and (5) comparing the degree of GAD65 autoantibody binding activity in the first and second serum samples to detect the presence or risk of type 1 diabetes in the subject.

In certain embodiments of the method, the control GAD65 polypeptide is wild-type GAD65 or is the corresponding GAD65 polypeptide not having the E517P mutation. In other embodiments, the control GAD65 polypeptide has the amino acid sequence of SEQ ID NO: 12 at position 515-525. In yet other embodiments, the mutant GAD65 polypeptide is full-length and/or consists of the GAD65 wild-type amino acid sequence at positions other than position 517.

In another aspect of the present invention, an isolated Ian5 nucleic acid is provided that is (a) a nucleic acid encoding the rat Ian5(+) polypeptide of SEQ ID NO: 3, (b) a nucleic acid encoding the rat Ian5(lyp) polypeptide of SEQ ID NO: 4; or (c) the full length complement of either (a) or (b). In certain embodiments, the nucleic acid includes the nucleotide sequence of either SEQ ID NO: 1 or nucleotides 1-312 of SEQ ID NO: 2.

In yet another aspect, polypeptides relating to rat Ian5, including mutant Ian5 polypeptides, are provided. In some embodiments, the isolated polypeptide includes the amino acid sequence of SEQ ID NO: 3 or consists essentially of amino acids 1-84 of SEQ ID NO: 4. In certain embodiments where the Ian5 polypeptide consists essentially of amino acids 1-84 of SEQ ID NO: 4, the polypeptide further consists of 1-40 random amino acids adjacent and carboxy terminal to amino acid 84. In one exemplary embodiment, the Ian5 polypeptide consisting essentially of amino acids 1-84 of SEQ ID NO: 4 and further consisting of 1-40 random amino acids adjacent and carboxy terminal to amino acid 84 is the polypeptide having the amino acid sequence of SEQ ID NO: 4.

In still another aspect, the present invention provides polypeptides relating to a mutant of human Ian5 protein. The polypeptides consist essentially of amino acids 1-85 of SEQ ID NO: 6. In certain embodiments, the polypeptide can further consist of 1-40 random amino acids adjacent and carboxy terminal to amino acid 85.

In another aspect, anti-Ian5 antibodies are provided. In some embodiments, the antibody specifically binds to rat Ian5(+) polypeptide and is not immunologically cross-reactive with human Ian5 or mouse Ian5 polypeptide; in other embodiments, the antibody specifically binds to rat Ian5(lyp) polypeptide and is not immunologically cross-reactive with rat Ian5(+), human Ian5, or mouse Ian5 polypeptide. The antibody can be monoclonal antibody, a polyclonal antibody, a single chain antibody, a heavy chain antibody, an F(ab′)₂, F(ab′), or Fv fragment.

The present invention also provides an expression construct characterized by the following elements linked in operable combination: (1) a transcriptional promoter; (2) an Ian5 nucleic acid that is (a) a nucleic acid encoding the rat Ian5(+) polypeptide of SEQ ID NO: 3, (b) a nucleic acid encoding the rat Ian5(lyp) polypeptide of SEQ ID NO: 4, or (c) the full length complement of either (a) or (b); and (3) a transcriptional terminator. In certain embodiments, the nucleic acid includes a nucleotide sequence that is either SEQ ID NO: 1 or nucleotides 1-312 of SEQ ID NO: 2.

In a related aspect, the present invention provides a prokaryotic or eukaryotic cell transformed or transfected with any of the above expression constructs. The prokaryotic or eukaryotic cell can be, for example, a bacterial cell, a yeast cell, or a mammalian cell. In other related aspects, the present invention provides a vector that includes any of the above expression constructs, as well as an isolated isolated host cell comprising the vector.

In addition, the present invention provides a method for producing an Ian5 polypeptide, the method including (1) growing cells transformed or transfected with the above vector; and (2) isolating the Ian5 polypeptide from the cells. The cells can be, for example, bacterial cells, yeast cells, or mammalian cells.

In yet another aspect, an in vitro method is provided for identifying agonists or antagonists of an Ian5 pathway to identify candidates for type 1 diabetes drug development. The method includes the following steps: (1) administering a candidate compound to a first cell that expresses the Ian5 polypeptide; (2) administering the candidate compound to a second cell that does not express the polypeptide; and (3) determining whether the candidate compound produces a physiological change in the first cell relative to the second cell. The first and second cells can be, for example, mammalian cells such as, e.g., hematopoictic cells. Further, in certain embodiments of the method, the Ian5 polypeptide is either rat Ian5(+), rat Ian(lyp), human Ian5, or mouse Ian5. In other embodiments, the candidate compound stimulates or inhibits cell proliferation.

Also, methods are provided for developing gene therapy for type 1 diabetes. In certain embodiments, the method includes the following steps: (1) administering a nucleic acid comprising an Ian5 polynucleotide to a non-human mammal having a frameshift mutation in the Ian5 gene locus, where the frameshift mutation results in a truncated mutant Ian5 polypeptide consisting essentially of amino acids corresponding to amino acids 1-84 of SEQ ID NO: 4, and where the non-human animal has one or more clinical symptom of type 1 diabetes; and (2) determining whether the nucleic acid encoding the Ian5 polynucleotide produces an amelioration of one or more clinical symptom of diabetes. In various embodiments, the nucleic acid comprising the Ian5 polynucleotide can be any of the following: (a) a vector that includes an Ian5 polynucleotide encoding a wild-type Ian5 polypeptide, where the wild-type Ian5-encoding polynucleotide is flanked by regions that promote intrachromosomal homologous recombination; (b) a vector that includes, linked in operative combination, a transcription promoter, the wild-type Ian5-encoding polynucleotide as in (a), and a transcription terminator; (c) an antisense Ian5 polynucleotide hybridizable within a cell to a polynucleotide encoding the truncated mutant Ian5 polypeptide; or (d) a vector that includes, linked in operative combination, a transcription promoter, the antisense Ian5 polynucleotide as in (c), and a transcription terminator. Also, in other embodiments, the non-human mammal is a DR.lyp/lyp rat or is a genetically modified mammal having an exogenous mutant Ian5 gene.

In yet other embodiments, the method for developing gene therapy for type 1 diabetes, includes the following steps: (1) administering a vector comprising an Ian5 polynucleotide to a non-human mammal having a knockout mutation in the Ian5 gene locus, where the non-human animal exhibits one or more clinical symptom of type 1 diabetes; and (2) determining whether the vector produces an amelioration of one or more clinical symptom of diabetes. In various embodiments, the vector comprising the Ian5 polynucleotide can be either of the following: (a) a vector that includes an Ian5 polynucleotide encoding a wild-type Ian5 polypeptide, said wild-type Ian5-encoding polynucleotide flanked by regions that promote intrachromosomal homologous recombination; or (b) a vector that includes, linked in operative combination, a transcription promoter, the wild-type Ian5-encoding polynucleotide as in (a), and a transcription terminator. In other embodiments, the non-human animal having the Ian5 knockout mutation is a transgenic knockout mouse.

In still another aspect, the present invention provides a method for detecting in a subject the presence of or risk of developing type 1 diabetes by either detecting an Ian5 mutation or detecting the level of Ian5 expression. In some embodiments, the method includes the steps of detecting the presence of a mutation at one or more nucleotide positions in the Ian5 gene in a sample from the subject, and therefrom identifying the presence or risk of developing type 1 diabetes. In certain embodiments, the mutation is a frameshift mutation resulting is a truncated mutant Ian5 polypeptide. The frameshift mutation can be, for example, a mutation in codon 85 of the human Ian5 coding sequence. The presence of the mutation can be detected by direct sequencing, hybridization with oligonucleotide probes, a ligation reaction, a polymerase chain reaction, or single nucleotide primer-guided extension assays.

In other embodiments, the method for detecting the presence of or the risk of developing type 1 diabetes includes the steps of (a) obtaining from the subject a biological sample containing or derived from lymphocytes; (b) obtaining a control sample containing or derived from lymphocytes; (c) determining the level of Ian5 gene expression in the subject sample and the control sample; and (d) comparing the level of Ian5 gene expression in the subject sample and the control sample to detect the presence of or the risk of developing type 1 diabetes. In certain embodiments, the level of Ian5 gene expression is determined with a nucleic acid probe or with an anti-Ian5 antibody.

Also, in another aspect, the present invention provides a method for identifying a genetic mutation that correlates with type 1 diabetes. The method includes the following steps: (a) determining the sequence of the Ian5 gene from a plurality of humans known to have diabetes; (b) comparing the sequence to the wild-type human Ian5 gene sequence; and (c) identifying mutations in the human Ian5 genes that correlate with the presence of type 1 diabetes.

### BRIEF DESCRIPTION OF THE DRAWINGS

FIG. 1. Comparison analysis between wild type GAD65 (upper panel) and E517P-GAD65 (lower panel) in GAD65Ab binding assays by using (A) monoclonal polyclonal Group A antibodies, or (B) peptide specific polyclonal Group B antibodies, and (C) 10 standard type 1 diabetes Group C sera. Four antibody dilutions (1:50 to 1:400) in each sample were included in the assay.

FIG. 2. Analysis of GAD65Ab binding of E517P-GAD65 in five groups of serum samples (A). Type 1 diabetic patients (n=95) and Type 2 patients (n=28), GAD65Ab positive healthy subjects (n=54), along with first degree relatives (n=36) and SMS samples (n=5) were included. The GAD65/67Ab double positive in type 1 and type 2 diabetes patient samples are highlighted by circles. One SMS sample (SW) exhibited differently from other SMS (n=4) in the binding assay by serum dilution study (1:500 to 1:8000) (B).

FIG. 3. A plot of E517P-GAD65 in percentage of GAD65 binding against GAD67 index among four groups of serum samples.

FIG. 4. Secondary structure prediction of peptide sequences of amino acid positions 505-544 in GAD65 and the corresponding sequence from GAD67. The GAD65 mutants GAD65-P (E517P) and GAD65-GVP (T515G, L516V and E517P) are shown with the GAD67 amino acid residue in bold, capital letters. Alpha helix sequences are indicated as 93a94 and motifs are roman numeral I, II, and LII. The E517P GAD65 mutation is sufficient to loose the 93194 alpha helix motif.

FIG. 5A. Physical map of the rat lyp gene region with genetic markers integrated (top). Overlapping PAC clones are shown along with the locations of genetic markers used to narrow the lyp interval, clone end STS assays, and the limits of the lyp interval itself as a red arrow at both top and bottom. Distances between markers may not be strictly to scale because they are estimated on STS-content. The lower part shows an expanded view of the lyp interval, showing the locations of known genes and the extent of the assembled sequence contigs of rat genomic DNA, along with the framework of mouse genomic DNA sequence. A 13 kb-long rat genomic sequence contig includes the rat Ian5 gene. Position coordinates shown are those from mouse sequence supercontig Mm6_WIFeb01_{—}100.

FIG. 5B. The cluster of Ian-related genes. In human, this gene family is present on chromosome 7q36.1. In the mouse, it is located on proximal chromosome 6. The position of the human and mouse orthologs of the LR8/Clastl (mouse accession no. AB031386) gene are also indicated as location aids although this gene is not in the IAN family. Various alternative names associated with each gene are indicated, and provisionally named previously unnamed members of the family as follows (these are indicated by underlines): For those genes without a common name, the Ian gene nomenclature is used. To avoid confusion, the genes in this family are referred to herein by using the name of the mouse ortholog and a prefix “h”, “m” or “r” to specify which species is indicated (e.g. “hIan2” for the human ortholog of mouse Ian2, otherwise known as himapl). Genes given the same Ian designation in different species have been determined to be orthologs of each other. Genes with different designations do not show enough similarity to be deemed orthologs, with the exception of hIan 7, which is orthologous to both mIan7 and mIan3. Genes in species without a clear ortholog in the other have been given unique Ian numbers (for example hIanl2). Positions shown are within the respective contigs (accession numbers NT_{—}007704.81Hs7-7861 for human and supercontig Mm6 W1Feb01-100 for mouse).

FIG. 5C. Diagram of the rat, mouse, and human Ian5 gene transcripts, with exon structure shown to scale. Beneath each transcript diagram is a diagram of the extent of the major ORF (the Ian5 coding region).

FIG. 6. Sequence of the BB rat Immune Associated Nucleotide (Ian5) gene. (A) A representative sequencing trace of DNA from BBDP/WorAp compared to wildtype BBDR/WorAp and F344 rats. The frameshift mutation at nucleotide position 473 in the DP rat DNA is indicated. DNA sequences were determined on an ABI PRISM® 3700 DNA Analyzer (Applied Biosystems, Foster City, Calif.) and analyzed by the using Phred, Phrap Consed and PolyPhred for sequence assembly and identification of sequence variants. (B) Nucleotide coding sequence of the rIan5 (+) gene (SEQ ID NO: 1). (C) Corresponding nucleotide sequence of the rIan5(lyp) gene; the single base pair deletion is indicated by the asterisk (*) (SEQ ID NO: 2).

FIG. 7. Sequence comparisons between BB DR wild-type (+/+) (SEQ ID NO: 3), lyp/lyp rat (SEQ ID NO: 4), mouse (SEQ ID NO: 5) and human (SEQ ID NO: 6) Immune Associated Nucleotide −5 (Ian5) and mouse Ian4 (SEQ ID NO: 7) predicted amino acid sequences. The deletion of a nucleotide in the codon for amino acid 85 of the rIan5 (lyp) changes the predicted downstream amino acids to include 19 amino acids (boxed) before the premature STOP codon at amino acid position 104. Putative ATP/GTP binding sites are boxed/shadowed and a hydrophobic putative transmembrane region underlined.

FIG. 8. Expression of rat Ian5 in tissues from lyp/lyp, lyp/+ and +/+DR BB rats.

FIG. 8A. Northern blot containing three μg of polyA+ RNA from thymus, spleen or kidney of each of +/+ or lyp/lyp rats probed with a 695 bp region of Ian5 showing a 1.4 kb transcript (Ian5). The blot was stripped and reprobed with a 1420 bp GAPDH probe (GAPDH). The images were quantitated using a phosphorimager and software and normalized to GAPDH expression in each lane. Size markers are indicated.

FIG. 8B. Northern blot containing three μg of polyA+ RNA from thymus, spleen, lymph node, and kidney from each of +/+, +/lyp, and lyp/lyp rats probed as in A. Size markers are indicated.

FIG. 8C. Methylene blue stain of the blot in panel b before probing showing even loading of 18S ribosomal RNA in each lane.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar to those described herein can be used in the practice or testing of the present invention, only exemplary methods and materials are described. For purposes of the present invention, the following terms are defined below.

The terms “a,” “an,” and “the” as used herein are not limiting and shall include plural referents, unless the context clearly indicates otherwise.

The term “type 1 diabetes” as used herein refers to the disease that exhibits the symptoms of insulin-dependent diabetes mellitus (IDDM). For example, where it is desirable to determine whether or not a subject falls within clinical parameters indicative of type 1 diabetes, signs and symptoms of type 1 diabetes that are accepted by those skilled in the art may be used to so designate a subject. A phenotypic trait, symptom, mutation or condition “correlates” with type I diabetes if it is repeatedly observed in individuals diagnosed as having some form of type 1 diabetes, or if it is routinely used by persons of ordinary skill in the art as a diagnostic criterion in determining that an individual has type 1 diabetes or a related condition.

“Isolating” a substance refers to removing a material from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acids could be part of a vector and/or such nucleic acids or polypeptides could be part of a composition, and still be isolated in that such vector or composition is not part of its natural environment.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions). Specifically, degenerate codon substitutions may be achieved by generating sequences in which the third position of one or more selected (or all) codons is substituted with mixed-base and/or deoxyinosine residues (see, e.g., Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, and mRNA encoded by a gene.

“Ian5 nucleic acids” refers to polynucleotides from the Ian5 gene locus, such as those encoding Ian5 polypeptides, including mRNAs, DNAs, cDNAs, antisense, and fragments, derivatives, and analogs thereof. In particular emodiments, Ian5 nucleic acids includes mutants encoding truncated forms of Ian5 polypeptides (see infra). Useful fragments and derivatives generally include those based on all possible codon choices for the same amino acid, and codon choices based on conservative amino acid substitutions. Other useful derivatives include, e.g., those having at least 60% polynucleotide sequence identity, typically at least 70%, more typically at least 80%, preferably 90%, and more preferably at least 95% sequence identity to the nucleic acids having the sequence set forth in SEQ ID NO: 1 or SEQ ID NO: 2.

“At least one mutation” as used herein means the substitution, addition, or deletion of at least one nucleotide anywhere in a nucleic acid as compared to the corresponding wild-type nucleotide sequence. A “point mutation” is a mutation within a nucleotide sequence that results in a change from one nucleotide to another. A “frameshift mutation” is a mutation caused by the insertion or deletion of one or more nucleotides so that the reading frame of codons in an mRNA molecule is altered during protein synthesis. Frame shift mutations cause an abnormal amino acid sequence to be translated beginning at the mutation site.

As used herein, “expression” of a gene or nucleic acid encompasses not only cellular gene expression, but also the transcription and translation of the nucleic acid in cloning systems and in any other context.

As used herein a “nucleic acid probe” is defined as a nucleic acid capable of binding to a target nucleic acid (e.g., an Ian5 nucleic acid) of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions.

Nucleic acid probes can be DNA or RNA fragments. DNA fragments can be prepared, for example, by digesting plasmid DNA, by use of PCR, or by synthesis via either the phosphoramidite method described by Beaucage and Carruthers (Tetrahedron Lett. 22:1859-1862 (1981)) or the triester method according to Matteucci, et al. (J. Am. Chem. Soc., 103:3185 (1981)). A double stranded fragment may then be obtained, if desired, by annealing the chemically synthesized single strands together under appropriate conditions, or by synthesizing the complementary strand using DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

A “labeled nucleic acid probe” is a nucleic acid probe that is bound, either covalently, through a linker, or through ionic, van der Waals or hydrogen bonds to a label such that the presence of the probe may be detected by detecting the presence of the label bound to the probe.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

“Ian5 polypeptide” or “GAD65 polypeptide” refer to a polypeptide encoded by the Ian5 or GAD65 gene locus, and fragments, derivatives, or analogs thereof. A “fragment” refers to a portion of a polypeptide typically having at least 10 contiguous amino acids, more typically at least 20, and still more typically at least 50 contiguous amino acids of the polypeptide. A “derivative” is a polypeptide having conservative amino acid substitutions as compared with another sequence. Derivatives further include, e.g., gycosylations, acetylations, phosphorylations, and the like. Ian5 or GAD65 polypeptides can also include polypeptides having one or more analogs of an amino acid (e.g., unnatural amino acids and the like), polypeptides with substituted linkages, as well as other naturally or non-naturally-occurring modifications known in the art. Such polypeptides will commonly be at least about 60% identical to the native Ian5 or GAD65 amino acid sequence, typically at least about 80%, more typically at least about 90%, and preferably at least about 95% identical. Unless the context clearly indicates otherwise, “Ian5 polypeptides” includes truncated mutant Ian5 polypeptides as further defined herein.

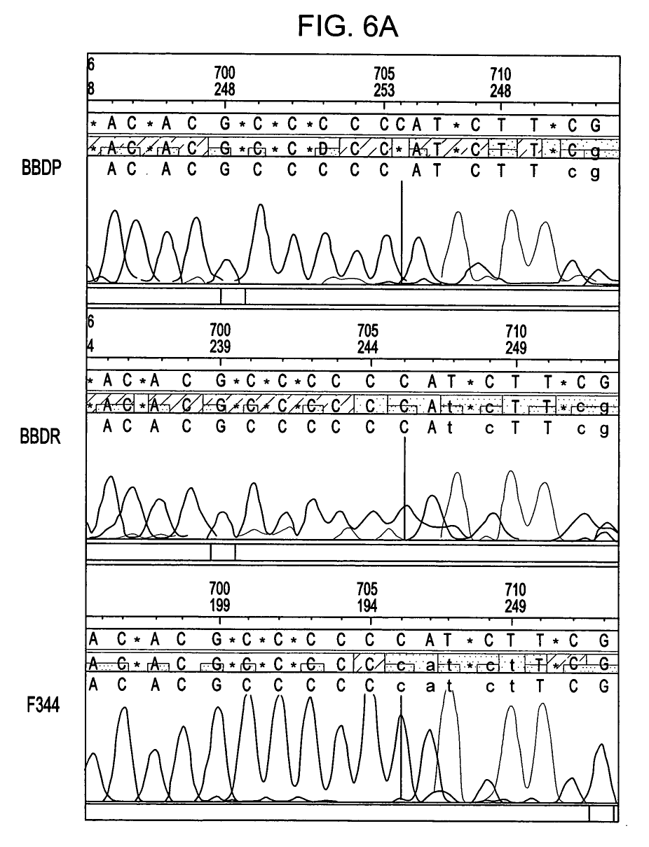

In the context of Ian5 nucleic acids and polypeptides, “correspondence” to another sequence (e.g., regions, fragments, nucleotide or amino acid positions, or the like) is based on the convention of numbering according to nucleotide or amino acid position number, and then aligning the sequences in a manner that maximizes the number of nucleotides or amino acids that match at each position. For example, a non-rat Ian5 amino acid sequence as provided herein may correspond to a rat Ian5 amino acid sequence according to the convention for numbering the rat Ian5 sequence as shown in FIG. 7, whereby a non-rat sequence is aligned with the rat Ian5 sequence such that at least 50%, typically at least 60%, more typically at least 70%, preferably at least 80% and more preferably at least 90% of the nucleotides in a given sequence of at least 20 consecutive nucleotides of a sequence are identical. Because not all positions with a given “corresponding region” need be identical, non-matching positions within a corresponding region are herein regarded as “corresponding positions.”

A “truncated mutant Ian5 polypeptide” means an Ian5 polypeptide that lacks at least amino acids carboxy-terminal to a position corresponding to amino acid 124 of SEQ ID NO: 3. (E.g., a truncated mutant form of human Ian5 polypeptide would lack at least amino acids carboxy-terminal to position 125 of SEQ ID NO: 6, based on the sequence alignment for maximum correspondence shown in FIG. 7.) A “truncated mutant Ian5 polypeptide” will also include at least amino acids corresponding to positions 1-64 of SEQ ID NO: 3. (E.g., for a human Ian polypeptide of SEQ ID NO: 6, a truncated mutant polypeptide would include amino acids 1-65.) Truncated mutant Ian5 polypeptides can also include 1-40 random carboxy-terminal amino acids.

In the context of the E517 GAD65 mutants described herein, a “corresponding GAD65 polypeptide not having the E517 mutation” means a GAD65 polypeptide that is identical to a particular E517 GAD65 mutant at all amino acid positions other than position 517.

The terms “identity” or “percent identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using either a PILEUP or BLAST sequence comparison algorithm (see, e.g., Higgins and Sharp, CABIOS 5:151-153 (1989); Altschul et al., J. Mol. Biol. 215:403-410 (1990)). Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see, generally, Ausubel et al., supra).

The phrase “substantially similar,” in the context of two nucleic acids or polypeptides, refers to two or more sequences or subsequences that have at least 60%, preferably 80%, most preferably 90-95% nucleotide or amino acid residue identity, when compared and aligned for maximum correspondence, as measured using either a PILEUP or BLAST sequence comparison algorithm (see, e.g., Higgins and Sharp, et. al.; Altschul et al., supra). Preferably, the substantial similarity exists over a region of the sequences that is at least about 50 residues in length, more preferably over a region of at least about 100 residues, and most preferably the sequences are substantially similar over at least about 150 residues. In a most preferred embodiment, the sequences are substantially similar over the entire length of the coding regions.

The phrase “hybridizing specifically to” as used herein refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

“Stringent hybridization conditions” and “stringent hybridization wash conditions” in the context of nucleic acid hybridization experiments, such as Southern and northern hybridizations, are sequence dependent, and are different under different environmental parameters. Longer sequences hybridize specifically at higher temperatures. An extensive guide to the hybridization of nucleic acids is found in Tijssen, Laboratory Techniques in Biochemistry and Molecular Biology: Hybridization with Nucleic Acid Probes (Elsevier, N.Y., 1993) (part I, chapter 2, “Overview of principles of hybridization and the strategy of nucleic acid probe assays”). Generally, highly stringent hybridization and wash conditions are selected to be about 5° C. lower than the thermal melting point (Tₘ) for the specific sequence at a defined ionic strength and pH. Typically, under “stringent conditions,” a probe will hybridize to its target subsequence, but to no other sequences.

A “conservative substitution,” when describing a protein, refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well known in the art (see also, e.g., Creighton, Proteins (W.H. Freeman and Company, 1984)). In addition, individual substitutions, deletions, or additions which alter, add, or delete a single amino acid or a small percentage of amino acids in an encoded sequence are also “conservatively modified variations.”

“Antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes IgG, IgM, IgA, IgD, and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}l by a disulfide bond. The F(ab)′₂ may be reduced under mild conditions to break the disulfide linkage in the hinge region, thereby converting the F(ab)′₂ dimer into an Fab′ monomer. The Fab′ monomer is essentially a Fab with part of the hinge region (see Fundamental Immunology (W. E. Paul ed., Raven Press 1993)). While various antibody fragments are defined in terms of the digestion of an intact antibody, such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fᵥ).

Two or more polypeptides are “immunologically cross-reactive” when the polypeptides specifically bind to the same antibody. Thus, polypeptides are typically immunologically cross-reactive where, e.g., the two polypeptides differ only by conservative substitutions.

The phrase “specifically (or selectively) binds to an antibody” or “specifically (or selectively) immunoreactive with,” when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised to the protein with the amino acid sequence encoded by any of the polynucleotides of the invention can be selected to obtain antibodies specifically immunoreactive with that protein and not with other proteins except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein (see, e.g., Harlow and Lane, Using Antibodies: A Laboratory Manual (Cold Spring Harbor Publications, New York, 1999) (“Harlow and Lane”) for a description of immunoassay formats and conditions that can be used to determine specific immunoreactivity). Typically, a specific or selective reaction will be at least twice background signal or noise and more typically more than 10 to 100 times background.

The term “decreased specific binding,” in reference to an antibody binding to a polypeptide, means a reduction in antibody binding of at least 10%, typically at least 20%, more typically at least 30%, and still more typically at least 40% when compared to the binding of the same antibody to a second polypeptide, as measured using standard immunoassays known in the art (e.g., ELISA, radioimmunoassay, and the like). In certain embodiments of the present invention, decreased specific binding of an antibody to a polypeptide is preferably at least 50%, more preferably at least 60%, still more preferably at least 70%, and even more preferably at least 80%, at least 85%, or at least 90% when compared to the binding of the same antibody to a second polypeptide.

The term “substantially the same specific binding activity” in the context of two polypeptides binding to an the same antibody means that each of the polypeptides are immunologically cross-reactive with the antibody and that each exhibits at least 90%, typically at least 95%, still more typically at least 98%, and preferably at least 99% or up to 100% of the specific binding activity as compared to the other polypeptide, as measured using standard immunoassays (e.g., ELISA, radioimmunoassay, and the like).

The term “C-terminal conformational epitope” as used herein refers to an epitope of GAD65 that is recognized by any of the GAD6, MICA-1, MICA-3, MICA-4, or MICA-6 monoclonal antibodies.

The term “genetically modified” or “transgenic” in the context of non-human animals means an animal whose genome has been altered by the inclusion of exogenous genetic material (a “transgene.” The “genetically modified” or “transgenic” animals can include those that contain cells or tissues with different genotypes (“chimerics”).

“Amelioration,” “treatment,” or “therapy” of a disease or disorder as used herein are synonymous and refer to slowing, stopping, or reversing progression of the disease, as evidenced by a reduction or elimination of either clinical or diagnostic symptoms. The terms also include complete or partial prevention of the occurrence or onset of a disease or disorder or some or all of the its symptoms.

In one aspect, the invention provides for GAD65 polypeptides having a non-alanine mutation at amino acid position 517 and lacking a GAD65 conformational epitope. The GAD65 mutants exhibit a decrease, as compared to a corresponding GAD65 polypeptide lacking the mutation, in specific binding to antibodies directed to a C-terminal conformational epitope of GAD65 (e.g., the GAD6, MICA-1, MICA-3, MICA-4, or MICA-6 monoclonal antibody). Typically, the mutant GAD65 polypeptide also exhibits decreased specific binding to an antibody directed to an epitope mapped to amino acids 506-531 of GAD65 (e.g., the MICA-2 MAb). In a particular embodiment, the mutation at position 517 is a glutamate to proline substitution (E517P). The mutant GAD65 proteins are useful, for example, in diagnostic methods as further described infra.

The mutant GAD65 polypeptides of the present invention comprise GAD65 amino acid sequences, or fragments, derivatives, or analogs thereof, sufficient such that the corresponding GAD65 polypeptide which lacks the E517 mutation will maintain immunological cross-reactivity with an antibody directed to the C-terminal conformational epitope of wild-type GAD65. The corresponding GAD65 antibody need not have 100% immunological cross-reactivity with the antibody directed to the C-terminal conformational epitope of wild-type GAD65, provided that the antibody binds specifically to the corresponding GAD65 polypeptide. Typically, the corresponding GAD65 polypeptide will have at least 70%, more typically at least 80%, still more typically at least 90%, preferably at least 95%, more preferably at least 98%, and even more preferably up to 100% immunological cross-reactivity with the antibody directed to the C-terminal conformational epitope of wild-type GAD65. In addition to fragments, mutant GAD65 polypeptides can comprise additions of one or more amino acids (e.g., at either the amino or carboxy terminus).

In certain embodiments, the mutant GAD65 polypeptide is a full-length GAD65 polypeptide. In other embodiments, the mutant GAD65 polypeptide comprises a sequence at position 515-525 that is either GVPDNEERMSR (SEQ ID NO: 15) or TLPDNEERMSR (SEQ ID NO: 16). In a particular embodiment, the mutant GAD65 polypeptide is a full-length GAD65 having the wild-type amino acid sequence at all positions except position 517.

The mutant GAD65 polypeptides, and corresponding GAD65 polypeptides lacking the E517 mutation, can be produced by various methods known in the art. The manipulations which result in their production can occur at the gene or protein level. For example, expression cloning, genomic cloning, and PCR (see, e.g., Sambrook et al., supra; Ausubel et al., supra.) can be used to obtain GAD65 polynucleotides (e.g., wild-type). Cloned GAD65 nucleic acids can be modified by any of numerous strategies known in the art (see, e.g., Sambrook et al., supra; Ausubel et al., supra) to generate a nucleic acid coding for a GAD65 polypeptide having a mutation at position 517 and, optionally, additional modifications such as, e.g., conservative substitutions, deletions, additions, insertions, and the like. For example, GAD65 nucleotide sequences can be modified to prepare sequences encoding the desired mutant GAD65 polypeptides using, e.g., standard in vitro site-directed mutagenesis (see, e.g., Hutchison et al., J. Biol. Chem. 253:6551-60 (1978)), the use of TAB® linkers (Pharmacia), PCR-mediated mutagenesis, and the like.

Other manipulations of the GAD65 polypeptides can also be made at the polypeptide level. Included within the scope of the invention are derivatives or analogs of GAD65 E517 mutants which are differentially modified during or after synthesis (e.g., in vivo or in vitro translation). Such modifications include conservative substitution, glycosylation, acetylation, phosphorylation, amidation, derivatization by known protecting/blocking groups, proteolytic cleavage, linkage to an antibody molecule or other cellular ligand, and the like. Any of numerous chemical modifications can be carried out by known techniques, including, but not limited to, specific chemical cleavage (e.g., by cyanogen bromide), enzymatic cleavage (e.g., by trypsin, chymotrypsin, papain, V8 protease, and the like); modification by, for example, NaBH₄ acetylation, formylation, oxidation and reduction, or metabolic synthesis in the presence of tunicamycin, and the like.

GAD65 polypeptides can be isolated and purified by standard methods including chromatography (e.g., ion exchange, affinity, sizing column chromatography, high pressure liquid chromatography), centrifugation, differential solubility, or by any other standard technique for the purification of proteins. The functional properties can be evaluated using any suitable assay as described herein or otherwise known to the skilled artisan. The GAD65 polypeptides can also be synthesized by standard chemical methods known in the art (see, e.g., Hunkapiller et al., Nature 310:105-11 (1984); Stewart and Young, Solid Phase Peptide Synthesis (Pierce Chemical Co., 2d ed. 1984)).

Specific binding of the polypeptides to antibodies directed to a GAD65 C-terminal conformational epitope can be evaluated using any suitable immunoassays, as described herein or otherwise known to the skilled artisan, which maintain the conformational epitope (e.g., radioimmunoassay). For example, the GAD65 polypeptides can be expressed by in vitro coupled transcription translation and the immunoreactivity of the ³⁵S-labed molecules analyzed by RIA to determine antigen-antibody binding. ((See, e.g., Example 1) Typical antibodies useful for analyzing the mutant GAD65 polypeptides are the GAD6, MICA-1, MICA-2, MICA-3, MICA-4, or MICA-6 monoclonal antibodies.

In another aspect of the invention, methods are provided for using the mutant GAD65 polypeptides to detect the presence of or risk of type 1 diabetes or a related disorder. Subject samples are analyzed for autoantibody binding to the mutant GAD65 polypeptide and a GAD65 polypeptide that has immunological cross-reactivity with conformational epitopes of wild-type GAD65. Comparison of autoantibody binding to the two GAD65 proteins can detect binding of GAD65 antibodies to conformational epitopes, which can improve prediction of type 1 diabetes by increasing diagnostic specificity.

Biological samples containing antibodies (e.g., serum samples) are obtained from human subjects for analysis of GAD65 autoantibody binding to GAD65 polypeptides. A first sample is contacted with a mutant GAD65 as described above; a second sample is contacted with a “control” GAD65 protein which has immunological cross-reactivity with conformational epitopes of wild-type GAD65. In certain embodiments, the control GAD65 is a GAD65 polypeptide not having the E517 mutation and which has substantially the same GAD6-, MICA-1-, MICA-3-, MICA-4-, or MICA-6-specific binding activity as the corresponding GAD65 polypeptide. Samples contacted with the GAD65 proteins are incubated under conditions sufficient to allow specific, detectable binding of any antibodies to their antigenic determinants, and the samples are then analyzed for GAD65 autoantibody binding. The GAD65 autoantibody binding of the two samples are then compared. A reduction in autoantibody binding to the mutant GAD65 polypeptide relative to the control GAD65 polypeptide indicates the presence of or the risk of developing type 1 diabetes or a related disorder. Reductions in autoantibody binding indicative of the presence of or risk of developing type 1 diabetes are typically at least a 30% reduction (i.e., mutant GAD65 binding being 70% or less than that of the control), more typically at least 40%, at least 50%, at least 60%, at least 70%, or at least 80%, with reductions of up to 85%, up to 90%, or up to 95% also typical.

In certain embodiments, the mutant GAD65 is full-length. In a particular embodiment, the E517 mutation is a glutamate to proline substitution (E517P). The mutant GAD65 polypeptide can consist of wild-type GAD65 sequences at all positions other than 517 or, alternatively, at all positions other than 515-525. Alternatively, mutant GAD65 fragments, derivatives, or analogs thereof can be used where the corresponding GAD65 polypeptide which lacks the E517 mutation maintains immunological cross-reactivity with an antibody directed to the C-terminal conformational epitope of wild-type GAD65. In a particular embodiments, the mutant GAD65 polypeptide comprises a sequence at position 515-525 that is either GVPDNEERMSR (SEQ ID NO: 15) or TLPDNEERMSR (SEQ ID NO: 16). In an exemplary embodiment, the mutant GAD65 polypeptide is a full-length GAD65 having the wild-type amino acid sequence at all positions except position 517.

In another embodiment of the present invention, the control GAD65 polypeptide is the corresponding GAD65 polypeptide not having the E517 mutation. In yet other embodiments, the control GAD65 polypeptide has the amino acid sequence TLEDNEERMSR (SEQ ID NO: 12) at position 515-525. In one exemplary embodiment, the control GAD65 polypeptide is wild-type GAD65.

Immunoassay methods for analyzing autoantibody binding to the GAD65 polypeptides are known in the art. (See generally, e.g., Harlow and Lane, supra.) Immunoassay methods suitable for use in the present invention are those which maintain C-terminal conformational epitopes of GAD65. In typical embodiments, the GAD65 polypeptides are not bound to a solid support. For example, radioimmunoassay (e.g., Protein A-Sepharose mediated RIA) using radiolabeled protein (e.g., ³⁵S-labeled) can be used. Antibody-bound and free antigen can then be separated by, e.g., binding sample antibodies to an antibody-specific solid support (such as, e.g., Protein A-Sepharose). Alternatively, for example, sample antibodies can be bound to, e.g., plastic wells such as in an ELISA format. Captured GAD65 polypeptides can then be detected using directly or indirectly labeled anti-GAD65 antibodies that are cross-reactive with both the mutant and the control GAD65 polypeptides.

In another aspect, the invention relates to the discovery that a frameshift deletion in rat Ian5 gene, a novel member of the Immune-Associated Nucleotide (IAN) related gene family, causes lymphopenia and clinical onset of diabetes symptoms. The mutation was discovered by positional cloning of the Iddml/lyp locus of the diabetes prone BB (BBDP) rat, which has many important features as a model for type 1 diabetes, including the presence of a simple Mendelian trait, lymphopenia, that is associated with diabetes. The frameshift deletion in Ian5 results in truncation of a significant portion of the encoded protein and is absent in 37 other inbred strains that are non-lymphopenic and non-diabetic. Ian5 belongs to a new family of GTP-binding proteins that are implicated in immune response functions (see, e.g., Krucken et al., Biochem. Biophys. Res. Comm. 230:167-170 (1997)).

The Ian5 nucleic acids, polypeptides, antibodies, and related compositions as further described herein are useful, for example, in diagnostic methods for predicting type 1 diabetes or related disorders, as well as in screening methods for identifying agonists or antagonists of biological pathways that relate to lymphocyte development and disease.

The rat Ian5 gene was identified by positional cloning of the Iddml/lyp locus. Comparative mapping was used to determine the syntenic chromosomal region in the mouse. A mouse YAC contig was then constructed spanning the mouse Iddml/lyp region and gene fragment isolated from that interval were used as probes to isolate corresponding orthologous rat gene fragments by cross-species cDNA selection. Rat YAC clones containing the rat fragments were then isolated to construct a rat YAC contig spanning the rat Iddml/lyp region.

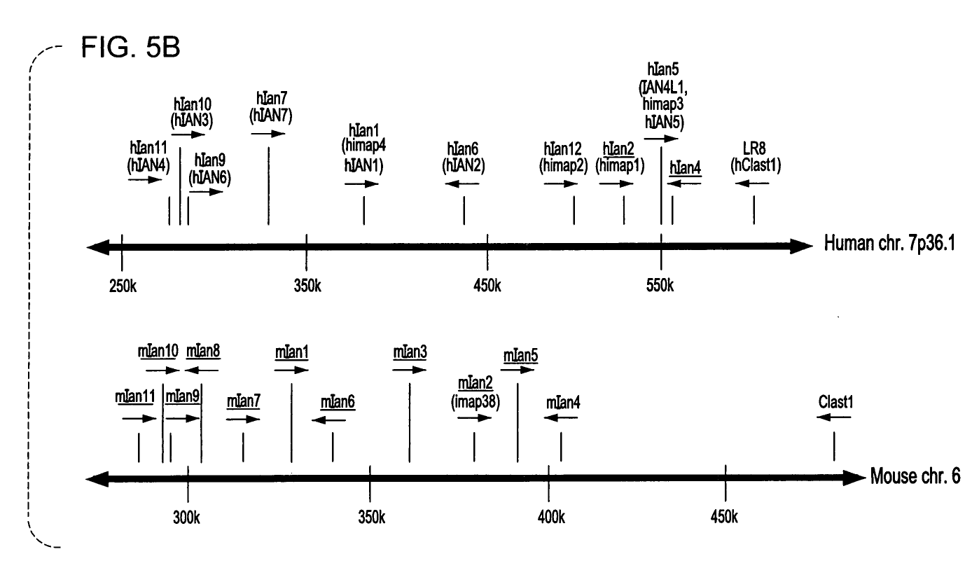

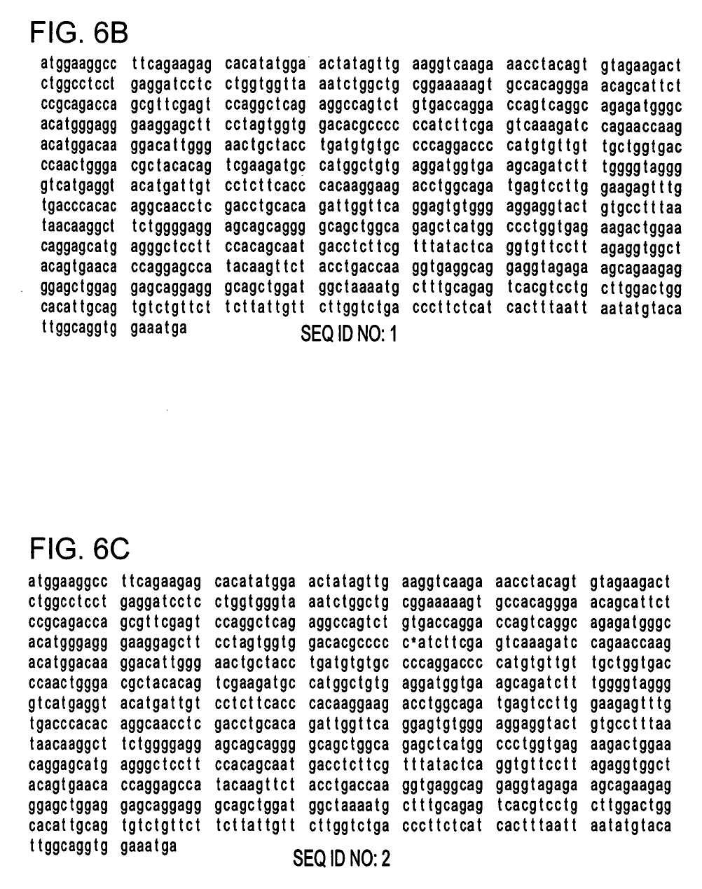

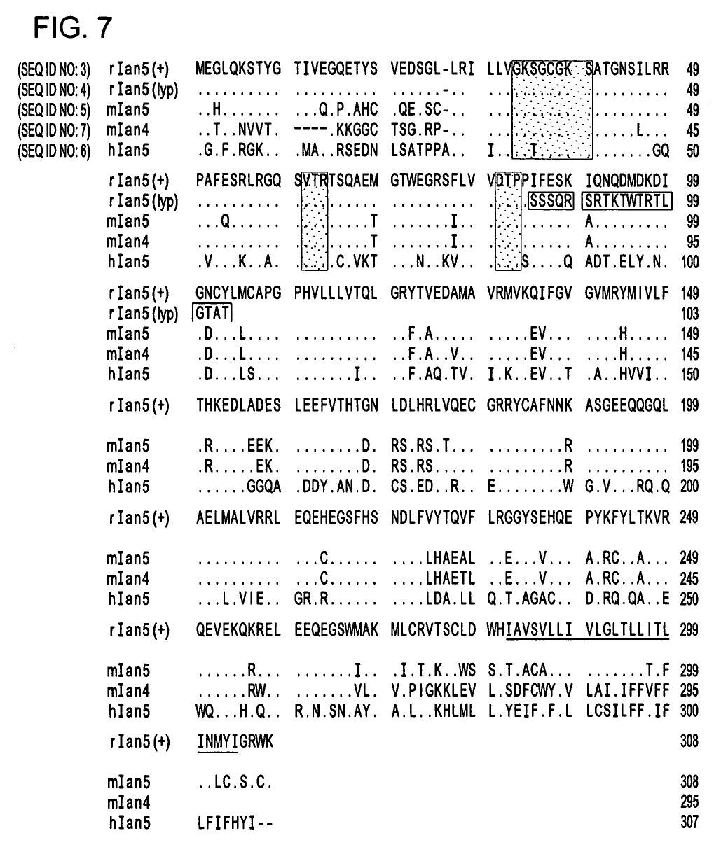

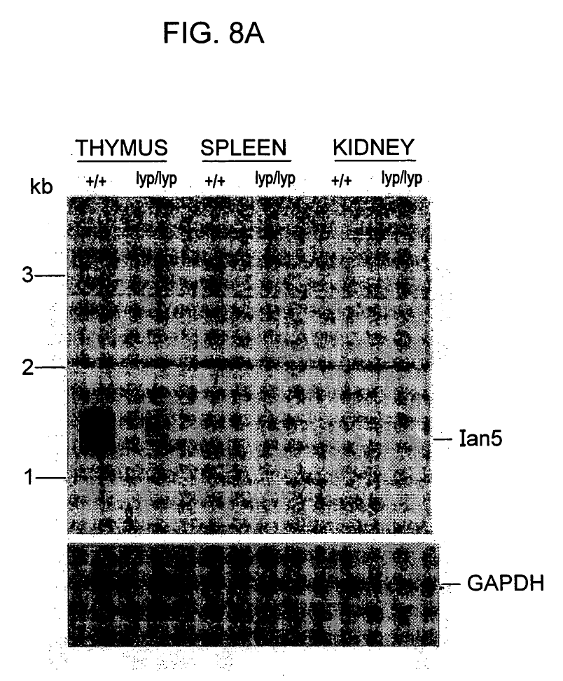

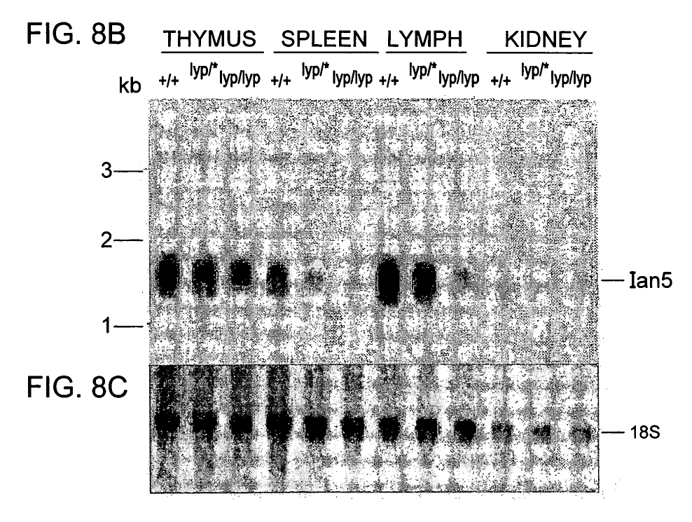

The wild-type (DR) sequence of rIan5 (herein rIan5(+)) predicts a protein with 308 amino acids, which is 80% identical to mIan5 and 52% identical to hIan5 (see Daheron et al., Nucleic Acids Res. 29:1308-1316 (2001); Stamm et al., Gene 282:159-167 (2002)). In the mutated rIan5 gene of the BBDP rat (herein rIan5(lyp)), a single nucleotide frameshift deletion at codon 84 (position 478 of the rIan5 gene) (see FIG. 6C and Table 4) changes the predicted downstream amino acids to include 19 amino acids before the premature stop codon (see FIG. 8).

In a specific embodiment, Ian5 nucleic acids comprise the nucleotide sequence of SEQ ID NO: 1, or the coding region of the rIan5(+) locus, or nucleic acid sequences (e.g., an open reading frame) encoding a rat Ian5(+) polypeptide (SEQ ID NO: 3). Ian5 nucleic acids further include mRNAs, genomic DNA, and antisense nucleic acids corresponding to the rIan5(+) locus.

In other embodiments, Ian5 nucleic acids comprise a nucleotide sequence coding for a truncated mutant Ian5 polypeptide. As defined herein, truncated mutant Ian5 polypeptides lack at least amino acids carboxy-terminal to a position corresponding to amino acid (aa) 124 of SEQ ID NO: 3 and include at least amino acids corresponding to positions 1-64 of SEQ ID NO: 3. In particular embodiments, the Ian5 nucleic acid encodes a truncated Ian5 polypeptide that lacks at least amino acids carboxy-terminal to a position corresponding to aa 104 of SEQ ID NO: 3; typically at least amino acids carboxy-terminal to a position corresponding to aa 94; and most typically at least amino acids carboxy-terminal to a position corresponding to aa 84. In other embodiments, the Ian5 nucleic acid encodes a truncated mutant Ian5 polypeptide that lacks at least amino acids carboxy-terminal to aa 74 of SEQ ID NO: 3 and/or that includes at least amino acids corresponding to positions 1-74 of SEQ ID NO: 3. In addition, sometimes from at least 1 and up to about 10, up to about 20, up to about 30, or up to about 40 carboxy-terminal amino acids encoded by the Ian5 nucleic acid can be random amino acids which do not have a corresponding region in a wild-type Ian5 sequence. The truncated mutant polypeptides encoded by the nucleic acids are typically mammalian (e.g., rat, human, mouse). Ian5 nucleic acids coding for truncated polypeptides can be constructed, e.g., from wild-type Ian5 gene sequences using known recombinant DNA methods for mutagenesis and cloning such as described in, e.g., Sambrook et al., supra; Ausubel et al., supra, or by identification of a mutation (e.g., frameshift mutation) in an Ian5 gene locus that results in a truncated Ian5 polypeptide, cloning of the mutant gene, and, if desired, further manipulation of the cloned gene using recombinant methods.

In an exemplary embodiment, the Ian5 nucleic acid coding for a truncated mutant Ian5 polypeptide comprises the nucleotide sequence of positions 1-312 of SEQ ID NO: 2, or the coding region of the rIan5(lyp) locus, or nucleic acid sequences (e.g., an open reading frame) encoding a rIan5(lyp) polypeptide. Ian5(lyp) nucleic acids further include mRNAs, genomic DNA, and antisense nucleic acids corresponding to the rIan5(lyp) locus.

Ian5 nucleic acids further include derivatives (e.g., nucleotide sequence variants), such as those encoding other possible codon choices for the same amino acid or conservative amino acid substitutions thereof, such as naturally occurring allelic variants. Due to the degeneracy of nucleotide coding sequences, other DNA sequences which encode substantially the same amino acid sequence as a Ian5 gene (e.g., the rIan5 (+) or rIan5(lyp) gene) can be used in the practice of the present invention. These include, but are not limited to, nucleotide sequences comprising all or portions of an Ian5 gene which is altered by the substitution of different codons that encode the same or a functionally equivalent amino acid residue (e.g., a conservative substitution) within the sequence, thus producing a silent change.

Ian5 nucleic acids further include those nucleic acids specifically hybridizable or complementary to the foregoing sequences. Hybridizable nucleic acids can comprise sequences complementary to at least 10, 25, 50, 100, 200, or 250 nucleotides or more of a Ian5 gene, including full-length complements of an Ian5 nucleic acid. Nucleic acids are specifically hybridizable to an Ian5 nucleic acid, or to a nucleic acid encoding a Ian5 derivative, under stringent conditions. Low, moderate, and high stringency conditions are well known to those of skill in the art and vary predictably depending on conditions such as salt concentrations, temperature, and the base composition of the particular nucleic acid sequence. (See, e.g., Sambrook et al., supra; Ausubel et al., supra.)

Various recombinant DNA methods known in the art can be used to prepare the Ian5 nucleic acids described hereinabove. For example, expression cloning, genomic cloning, and PCR (see, e.g., Sambrook et al., supra; Ausubel et al., supra.) can be used to obtain Ian5 polynucleotides (e.g., rIan5(+), rIan5(lyp), or Ian5 sequences from other species) which can be used for further manipulation. Nucleic acid sequences can also be produced by synthesis using standard methods (e.g., by use of a commercially available automated DNA synthesizer) (typically for shorter nucleic acids). Nucleic acids can be further manipulated as desired using routine techniques. (See generally, e.g., Sambrook et al., supra; Ausubel et al., supra.) For example, known Ian5 sequences can be modified to prepare sequences encoding truncated Ian5 polypeptides using, e.g., standard in vitro site-directed mutagenesis (see, e.g., Hutchison et al., J. Biol. Chem. 253:6551-60 (1978)), the use of TAB® linkers (Pharmacia), PCR mutagenesis methods, and the like.

The Ian5-encoding nucleic acids can be inserted into an appropriate expression vector (i.e., a vector which contains the necessary elements for the transcription and translation of the inserted polypeptide-coding sequence). A variety of host-vector systems can be utilized to express an Ian5 polypeptide-coding sequence. These include, for example, mammalian cell systems transfected with plasmid vectors or infected with virus (e.g., vaccinia virus, adenovirus, parvoviruses (e.g., AAV), sindbis virus, Venezuelan equine encephalitis (VEE) virus, and the like), insect cell systems infected with virus (e.g., baculovirus), microorganisms such as yeast containing yeast vectors, or bacteria transformed with bacteriophage DNA, plasmid DNA, or cosmid DNA. The expression elements of vectors vary in their strengths and specificities. Depending on the host-vector system utilized, any one of a number of suitable transcription and translation elements can be used. In specific embodiments, the Ian5 polypeptide is expressed in human cells, rat cells, other mammalian cells, yeast or bacteria.

Any suitable method can be used for insertion of Ian5 nucleic acids into an expression vector. Suitable expression vectors typically include appropriate transcriptional and translational control signals. Suitable methods include in vitro recombinant DNA and synthetic techniques and in vivo recombination techniques (genetic recombination). Expression of nucleic acid sequences can be regulated by a second nucleic acid sequence so that the encoded nucleic acid is expressed in a host transformed with the recombinant DNA molecule. For example, expression of an Ian5 polypeptide can be controlled by any suitable promoter/enhancer element known in the art. Suitable promoters include, for example, the SV40 early promoter region (Benoist and Chambon, Nature 290:304-10 (1981)), the promoter contained in the 3′ long terminal repeat of Rous sarcoma virus (Yamamoto et al., Cell 22:787-97 (1980)), the herpes thymidine kinase promoter (Wagner et al., Proc. Natl. Acad. Sci. USA 78:1441-45 (1981)), the cytomegalovirus promoter, the translational elongation factor EF-1α promoter, the regulatory sequences of the metallothionein gene (Brinster et al., Nature 296:39-42 (1982)), prokaryotic promoters such as, for example, the β-lactamase promoter (Villa-Komaroff et al., Proc. Natl. Acad. Sci. USA 75:3727-31 (1978)) or the tac promoter (deBoer et al., Proc. Natl. Acad. Sci. USA 80:21-25 (1983)), plant expression vectors including the cauliflower mosaic virus ³⁵S RNA promoter (Gardner et al., Nucl. Acids Res. 9:2871-88 (1981)), and the promoter of the photosynthetic enzyme ribulose biphosphate carboxylase (Herrera-Estrella et al., Nature 310:115-20 (1984)), promoter elements from yeast or other fungi such as the GAL7 and GAL4 promoters, the ADH (alcohol dehydrogenase) promoter, the PGK (phosphoglycerol kinase) promoter, the alkaline phosphatase promoter, and the like.

Other mammalian promoters include, for example, the following animal transcriptional control regions, which exhibit tissue specificity: the elastase I gene control region which is active in pancreatic acinar cells (Swift et al., Cell 38:639-46 (1984); Omitz et al., Cold Spring Harbor Symp. Quant. Biol. 50:399-409 (1986); MacDonald, Hepatology 7(1 Suppl.):42S-51S (1987); the insulin gene control region which is active in pancreatic beta cells (Hanahan, Nature 315:115-22 (1985)), the immunoglobulin gene control region which is active in lymphoid cells (Grosschedl et al., Cell 38:647-58 (1984); Adams et al., Nature 318:533-38 (1985); Alexander et al., Mol. Cell. Biol. 7:1436-44 (1987)), the mouse mammary tumor virus control region which is active in testicular, breast, lymphoid and mast cells (Leder et al., Cell 45:485-95 (1986)), the albumin gene control region which is active in liver (Pinkert et al., Genes Dev. 1:268-76 (1987)), the alpha-fetoprotein gene control region which is active in liver (Krumlauf et al., Mol. Cell. Biol. 5:1639-48 (1985); Hammer et al., Science 235:53-58 (1987); the alpha 1-antitrypsin gene control region which is active in the liver (Kelsey et al., Genes and Devel. 1: 161-71 (1987)); the beta-globin gene control region which is active in myeloid cells (Magram et al., Nature 315:338-40 (1985); Kollias et al., Cell 46:89-94 (1986); the myelin basic protein gene control region which is active in oligodendrocyte cells in the brain (Readhead et al., Cell 48:703-12 (1987)); the myosin light chain-2 gene control region which is active in skeletal muscle (Shani, Nature 314:283-86 (1985)); and the gonadotropic releasing hormone gene control region which is active in the hypothalamus (Mason et al., Science 234:1372-78 (1986)).

In certain embodiments, a vector is used that comprises, in operative combination, a transcription promoter, the Ian5-encoding nucleic acid, a transcription terminator, and one or more origins of replication. In other embodiments, the vector includes one or more selectable markers (e.g., an antibiotic resistance gene). Suitable selectable markers include, for example, those conferring resistance to ampicillin, tetracycline, neomycin, G418, and the like.

Once a suitable expression vector host system and growth conditions are established, methods that are known in the art can be used to propagate it. In addition, host cells can be chosen that modulate the expression of the inserted nucleic acid sequences, or that modify or process the gene product in the specific fashion desired. Expression from certain promoters can be elevated in the presence of certain inducers; thus, expression of the Ian5 sequence can be controlled. Furthermore, different host cells having characteristic and specific mechanisms for the translational and post-translational processing and modification (e.g., glycosylation or phosphorylation) of polypeptides can be used. Appropriate cell lines or host systems can be chosen to ensure the desired modification and processing of the expressed polypeptide. For example, expression in a bacterial system can be used to produce an unglycosylated polypeptide.

The invention further relates to the Ian5 polypeptides, including derivatives and analogs. The production and use of Ian5 polypeptides, and derivatives and analogs thereof, are also within the scope of the present invention. The Ian5 polypeptides, derivatives, and analogs of the present invention generally relate to rat Ian5 polypeptide sequences as well as truncated mutant polypeptides corresponding to Ian5 sequences from rat and other species, including mouse and human.

In one aspect, the invention provides the amino acid sequences of the rat Ian5(+) polypeptide (SEQ ID NO:3). In another aspect, the invention provides a truncated mutant Ian5 polypeptide which lacks at least amino acids carboxy-terminal to a position corresponding to amino acid (aa) 124 of SEQ ID NO: 3 and includes at least amino acids corresponding to positions 1-64 of SEQ ID NO: 3. Typcially, the truncated Ian5 polypeptide lacks at least amino acids carboxy-terminal to a position corresponding to aa 104 of SEQ ID NO: 3; typically at least amino acids carboxy-terminal to a position corresponding to aa 94; and most typically at least amino acids carboxy-terminal to a position corresponding to aa 84. In other embodiments, the truncated mutant Ian5 polypeptide will lack at least amino acids carboxy-terminal to aa 74 of SEQ ID NO: 3 and/or will include at least amino acids corresponding to positions 1-74 of SEQ ID NO: 3. In addition, 0, 1-10, 1-20, 1-30, or 1-40 carboxy-tenminal amino acids can be random amino acids which do not have a corresponding region in a wild-type Ian5 sequence. The truncated mutant polypeptides are typically mammalian (e.g., rat, human, mouse). In an exemplary embodiment, the truncated Ian5 polypeptide has the amino acid sequence of the rat Ian5(lyp) polypeptide (SEQ ID NO: 4).

In certain embodiments, the Ian5 polypeptides include derivatives and analogs of the polypeptides described herein. In specific embodiments, Ian5 polypeptide derivatives and analogs as well are functionally active (i.e., capable of exhibiting one or more functional activities associated with a full-length, wild-type rIan5(+) polypeptide or with a truncated mutant Ian5 polypeptide such as, e.g., rIan5(lyp)). As one example, truncated polypeptides or derivatives or analogs which have the desired immunogenicity or antigenicity, can be used, for example, in immunoassays, for immunization, for inhibition of Ian5 activity, and the like. Fragments, derivatives, or analogs that retain, or alternatively lack or inhibit, a desired Ian5 property of interest (e.g., binding to a Ian5 binding partner, GTPase activity, or modulation (e.g., inhibition or stimulation) of cell proliferation such as, e.g., hematopoietic cell proliferation) can be used as inducers or inhibitors of such a property and its physiological correlates. Significantly truncated polypeptides, derivatives, or analogs of an Ian5 polypeptide can be tested for the desired activity by procedures known in the art, including but not limited to the functional assays described herein.

Ian5 polypeptide derivatives include naturally-occurring amino acid sequence variants as well as those altered by substitution, addition, or deletion of one or more amino acid residues that provide for functionally active molecules. Ian5 polypeptide derivatives include, e.g., those containing as a primary amino acid sequence all or part of the amino acid sequence of an Ian5 polypeptide including altered sequences in which one or more functionally equivalent amino acid residues (e.g., a conservative substitution) are substituted for residues within the sequence, resulting in a silent change.

Derivatives or analogs of an Ian5 polypeptide include but are not limited to those molecules comprising regions that are substantially similar to the Ian5 polypeptide (e.g., in various embodiments, at least 50%, 60%, 70%, 75%, 80%, 90%, or 95% identity or similarity over an amino acid sequence of identical size) when compared to an aligned sequence in which the alignment is done by a computer sequence comparison/alignment program known in the art, or whose coding nucleic acid is capable of hybridizing to a Ian5 nucleic acid under high stringency conditions. Ian5 polypeptides further comprise derivatives having an antigenic determinant (e.g., can be recognized by an antibody specific for a rat Ian5). In specific embodiments, derivatives having an antigenic determinant recognized by an anti-rat Ian5 antibody are not immunologically cross-reactive with either human or mouse Ian5.

The Ian5 polypeptide derivatives and analogs can be produced by various methods known in the art. The manipulations which result in their production can occur at the gene or protein level. For example, the cloned Ian5 nucleic acids can be modified by any of numerous strategies known in the art (see, e.g., Sambrook et al., supra; Ausubel et al., supra), such as making conservative substitutions, deletions, additions, insertions, and the like. Manipulations of the Ian5 polypeptide sequence can also be made at the polypeptide level. Included within the scope of the invention are Ian5 polypeptide derivatives or analogs which are differentially modified during or after synthesis (e.g., in vivo or in vitro translation). Such modifications include conservative substitution, glycosylation, acetylation, phosphorylation, amidation, derivatization by known protecting/blocking groups, proteolytic cleavage, linkage to an antibody molecule or other cellular ligand, and the like. Any of numerous chemical modifications can be carried out by known techniques, including, but not limited to, specific chemical cleavage (e.g., by cyanogen bromide), enzymatic cleavage (e.g., by trypsin, chymotrypsin, papain, V8 protease, and the like); modification by, for example, NaBH₄ acetylation, formylation, oxidation and reduction, or metabolic synthesis in the presence of tunicamycin, and the like.

In a specific embodiment, the Ian5 derivative is a chimeric, or fusion, protein comprising an Ian5 polypeptide (e.g., rIan5(+) or truncated mutant Ian5 polypeptides such as, for example rIan5(lyp)) joined at its amino- or carboxy-terminus via a peptide bond to an amino acid sequence of a different protein. In one embodiment, such a chimeric protein is produced by recombinant expression of a nucleic acid encoding the protein. The chimeric product can be made by ligating the appropriate nucleic acid sequence, encoding the desired amino acid sequences, to each other in the proper coding frame and expressing the chimeric product by methods commonly known in the art. Alternatively, the chimeric product can be made by protein synthetic techniques (e.g., by use of an automated peptide synthesizer).

Ian5 polypeptides can be isolated and purified by standard methods including chromatography (e.g., ion exchange, affinity, sizing column chromatography, high pressure liquid chromatography), centrifugation, differential solubility, or by any other standard technique for the purification of proteins. The functional properties can be evaluated using any suitable assay as described herein or otherwise known to the skilled artisan, including, for example, binding to an Ian5 binding partner, modulation of cell proliferation, GTPase activity, or GDP/GTP-binding properties (see, e.g., Warner et al., J. Biol. Chem. 273:23976-23983 (2998); Carty et al., Methods Enzymol. 237:38-44 (1994), which describe guanine nucleotide binding assays). The protein can also be synthesized by standard chemical methods known in the art (see, e.g., Hunkapiller et al., Nature 310:105-11 (1984); Stewart and Young, Solid Phase Peptide Synthesis (Pierce Chemical Co., 2d ed. 1984)).

In a specific embodiment of the present invention, Ian5 polypeptides, whether produced by recombinant DNA techniques, by chemical synthetic methods, or by purification of native polypeptides, include but are not limited to those containing as a primary amino acid sequence the amino acid sequence of rIan5(+) polypeptide (SEQ ID NO: 3) and rIan5(lyp) (SEQ ID NO: 4), as well as derivatives and analogs thereof. Ian5 polypeptides and derivatives and analogs thereof can be used as an immunogen to generate antibodies which immunospecifically bind such Ian polypeptides and derivatives and analogs thereof. Such antibodies include but are not limited to polyclonal antibodies, monoclonal antibodies, chimeric antibodies, single chain antibodies, heavy chain antibody fragments (e.g., F(ab′), F(ab′)₂, Fv, or hypervariable regions), and an Fab expression library. In a specific embodiment, polyclonal and/or monoclonal antibodies to whole, intact rat Ian5 polypeptide (e.g., rIan5(+)) or to a truncated mutant Ian5 polypeptide (e.g., rIan5(lyp)) are produced. In another embodiment, antibodies to a domain of a rat Ian5 polypeptide or to a truncated mutant polypeptide are produced. Typically, the anti-rat Ian5 polypeptides of the present invention are not immunologically cross-reactive with an Ian5 polypeptide from another species (e.g., mouse or human). In a particular embodiment, antibodies to truncated mutant Ian5 polypeptides are not immunologically cross-reactive with the full-length polypeptide and vice versa. For example, anti-rIan5(lyp) antibodies can be produced that are not immunologically cross-reactive with rIan5(+); such antibodies are typically directed to, e.g., an amino acid sequence within the region comprising aa positions 85-103, although such antibodies can also be to conformational epitopes specific for one form of Ian5 polypeptide. In certain embodiments, fragments of an Ian5 polypeptide identified as hydrophilic are used as immunogens for antibody production.

Methods for making and using antibodies are known in the art. (See generally, e.g., Harlow and Lane, Using Antibodies: A Laboratory Manual (Cold Spring Harbor Laboratory, Cold Spring Harbor, N.Y., 1999.) Antibodies can be polyclonal or monoclonal. Polyclonal sera typically contain mixed populations of antibodies specifically binding to several epitopes along the length of an Ian5 polypeptide. However, polyclonal sera can be specific to a particular segment of the polypeptide, such as aa 85-105 of rIan5(lyp). For preparation of monoclonal antibodies directed toward an Ian5 polypeptide, or a derivative or analog thereof, any technique which provides for the production of antibody molecules by continuous cell lines in culture can also be used. Such techniques include, for example, the hybridoma technique (see, e.g., Kohler and Milstein, Nature 256:495-97 (1975)), the trioma technique, (see, e.g., Hagiwara and Yuasa, Hum. Antibodies Hybridomas 4:15-19 (1993)), the human B-cell hybridoma technique (see, e.g., Kozbor et al., Immunology Today 4:72 (1983)).

Antibodies can also be chimeric (see, e.g., U.S. Pat. Nos. 4,816,567; 4,816,397; 5,693,762; and 5,712,120; International Patent Publications WO 87/02671 and WO 90/00616; and European Patent Publication EP 239 400), humanized (see, e.g., Queen et al., Proc. Natl. Acad. Sci. USA 86:10029-10033 (1989) and WO 90/07861, U.S. Pat. No. 5,693,762, U.S. Pat. No. 5,693,761, U.S. Pat. No. 5,585,089, U.S. Pat. No. 5,530,101 and Winter, U.S. Pat. No. 5,225,539), or human (see, e.g., Lonberg et al., WO93/12227 (1993); U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741 (1991)). Several mouse antibodies of different binding specificities are available as starting materials for making humanized antibodies. In addition, the anti-Ian5 antibodies can be single chain antibodies (see, e.g., U.S. Pat. Nos. 4,946,778 and 5,969,108) or heavy chain antibodies (see, e.g., Muyldermans and Lauwereys, J. Mol. Recognit. 12:131-40 (1999); Arbabi Ghahroudi et al., FEBS Lett. 414:521-26 (1997)).

In the production of antibodies, screening for the desired antibody can be accomplished by techniques known in the art (e.g., ELISA (enzyme-linked immunosorbent assay)). In one example, a fragment of rIan5(lyp) containing amino acids 85-103 of SEQ ID NO: 4 can be used to assay generated hybridomas for a product which specifically binds to rIan5(lyp). Also, for selection of an antibody that specifically binds to a first Ian5 polypeptide (e.g., rIan5(lyp) or rIan5(+)) but which does not specifically bind a different Ian5 polypeptide (e.g., rIan5(+) or hIan5, respectively), one can select on the basis of antibody positive binding to the first Ian5 polypeptide and a lack of antibody binding to the second different Ian5 polypeptide.

Antibodies specific to a domain of Ian5 polypeptides are also provided. The foregoing antibodies can be used in methods relating to the localization and activity of the Ian5 polypeptide sequences of the invention (e.g., for imaging proteins, measuring levels thereof in appropriate physiological samples, in diagnostic methods, and the like).

Ian5 nucleic acids and polypeptides, including derivatives and analogs, also have uses in screening assays to detect candidate compounds that specifically bind to Ian5 nucleic acids or polypeptides or that otherwise affect upstream or downstream Ian5 biological pathways in a cell. The agonists are typically identified in vitro by cell-based and/or non-cell-based assays. These assays can be used to identify agents that are therapeutically effective (e.g., reduction of lymphopenia in subjects having diabetes) or as lead compounds for drug development.

In a typical in vitro cell-based assay, recombinant cells expressing Ian5 nucleic acids can be used to screen candidate compounds for those that affect Ian5 biological pathways in the cell. Effects on Ian5 pathways can include, for example, effects on Ian5 expression (e.g., transcription of mRNA, translation of the mRNA, synthesis of Ian5 polypeptides, effects on Ian5 polypeptide stability or localization), effects on Ian5 polypeptide function (e.g., GTPase activity), or other effects specific to an Ian5 pathway as determined by, e.g., examining differential physiological responses Ian5-expressing and non-Ian5-expressing cells. Such effects on Ian5 pathways can be identified as physiological changes, such as, for example, changes in cell growth rate, division, viability (e.g., apoptosis effects), phosphorylation of cellular proteins, activation of transcription factors (e.g., NF-Kb), expression of cell surface markers, Ca²⁺ flux, and the like. In one embodiment, candidate compounds are administered to recombinant cells expressing an Ian polypeptide to identify those compounds that produce a physiological change. The physiological change can be determined relative to control cells not expressing the Ian5 polypeptide. In another embodiment, the method comprises administering a candidate compound to a first cell that expresses a first Ian5 polypeptide; administering the candidate compound to a second cell that expresses a second Ian polypeptide; and determining whether the candidate compound modulates the activity of the first Ian5 polypeptide but not the activity of the second Ian5 polypeptide. For example, the first Ian5 polypeptide can be a truncated mutant Ian5 polypeptide (e.g., rIan5(lyp) or a corresponding human Ian5 mutant) and the second can be the wild-type Ian5 polypeptide (e.g., rIan5(+) or hIan5).

In another embodiment, the two-hybrid system for selecting interacting proteins in yeast (see, e.g., Fields and Song, Nature 340:245-46 (1989); Chien et al., Proc. Natl. Acad. Sci. USA 88:9578-82 (1991)) can be used to identify candidate compounds that specifically bind to an Ian5 polypeptide or derivative.

Candidate compounds can also be identified by non-cell-based in vitro screens. For example, recombinant cells expressing Ian5 nucleic acids can be used to recombinantly produce Ian5 polypeptide for in vitro assays to identify candidate compounds that bind to Ian5 polypeptide. Candidate compounds (such as putative binding partners of Ian5 or small molecules) are contacted with the Ian5 polypeptide (e.g., wild-type Ian5, a truncated mutant, or a derivative or analog thereof) under conditions conducive to binding, and then candidate compounds that specifically bind to the Ian5 polypeptide are identified. Similar methods can be used to screen for candidate compounds that bind to nucleic acids encoding Ian5. Methods that can be used to carry out the foregoing are commonly known in the art, and include diversity libraries, such as random or combinatorial peptide or non-peptide libraries that can be screened for candidate compounds that specifically bind to Ian5 polypeptide.

Many libraries are known in the art, including, for example, chemically synthesized libraries, recombinant phage display libraries, and in vitro translation libraries. Chemically synthesized libraries are described in, e.g., Fodor et al., Science 251:767-73 (1991); Houghten et al., Nature 354:84-86 (1991); Lam et al., Nature 354:82-84 (1991); Medynski, Bio/Technology 12:709-10 (1994); Gallop et al., J. Med. Chem. 37:1233-51 (1994); Ohlmeyer et al., Proc. Natl. Acad. Sci. USA 90:10922-26 (1993); Erb et al., Proc. Natl. Acad. Sci. USA 91:11422-26 (1994); Houghten et al., BioTechniques 13:412-21 (1992); Jayawickreme et al., Proc. Natl. Acad. Sci. USA 91:1614-18 (1994); Salmon et al., Proc. Natl. Acad. Sci. USA 90:11708-12 (1993); International Patent Publication WO 93/20242, and Brenner and Lemer, Proc. Natl. Acad. Sci. USA 89:5381-83 (1992). Recombinant phage display libraries are exemplified in, e.g., Scott and Smith, Science 249:386-90 (1990); Devlin et al., Science 249:404-06 (1990); Christian et al., J. Mol. Biol. 227:711-18 (1992); Lenstra, J. Immunol. Meth. 152:149-57 (1992); Kay et al., Gene 128:59-65 (1993); and International Patent Publication WO 94/18318. In vitro translation-based libraries include, e.g., those described in International Patent Publication WO 91/05058; and Mattheakis et al., Proc. Natl. Acad. Sci. USA 91:9022-26 (1994). By way of examples of nonpeptide libraries, a benzodiazepine library (see, e.g., Bunin et al., Proc. Natl. Acad. Sci. USA 91:4708-12 (1994)) can be adapted for use. Peptide libraries (see, e.g., Simon et al., Proc. Natl. Acad. Sci. USA 89:9367-71 (1992)) can also be used. Another example of a library that can be used is one in which the amide functionalities in peptides have been permethylated to generate a chemically transformed combinatorial library, such as described by Ostresh et al., Proc. Natl. Acad. Sci. USA 91:11138-42 (1994).

Screening of the libraries can be accomplished by any of a variety of commonly known methods. (See, e.g., Parmley and Smith, Adv. Exp. Med. Biol. 251:215-18 (1989); Scott and Smith, supra; Fowlkes et al., BioTechniques 13:422-28 (1992); Oldenburg et al., Proc. Natl. Acad. Sci. USA 89:5393-97 (1992); Yu et al., Cell 76:933-45 (1994); Staudt et al., Science 241:577-80 (1988); Bock et al., Nature 355:564-66 (1992); Tuerk et al., Proc. Natl. Acad. Sci. USA 89:6988-92 (1992); Ellington et al., Nature 355:850-52 (1992); U.S. Pat. Nos. 5,096,815, 5,223,409, and 5,198,346; Rebar and Pabo, Science 263:671-73 (1994); and International Patent Publication WO 94/18318.) For example, in one embodiment, screening can be carried out by contacting the library members with a Ian5 polypeptide (or nucleic acid or derivative) immobilized on a solid phase and harvesting those library members that bind to the polypeptide (or nucleic acid or derivative). Examples of such screening methods, termed “panning” techniques, are described by way of example in, e.g., Parmley and Smith, Gene 73:305-18 (1988); Fowlkes et al., supra; International Patent Publication WO 94/18318; and in references cited hereinabove.

In additional embodiments, candidate compounds can be further tested (for example, in secondary or tertiary screens) in vivo on animal disease models such as, e.g., animal models for type 1 diabetes or a related disorder. Compounds are administered to the animals either before or after onset of disease symptoms using one or more treatment regimens (based on, e.g., administration routes, dosage, frequency of dosing, etc.) and the animals are monitored for amelioration of one or more disease symptoms. In one particular embodiment, the candidate compounds can be screened for efficacy in ameliorating one or more symptoms of lymphopenia or type 1 diabetes in the BBDP or DR.lyp/lyp rat. In other embodiments, compounds are tested on a genetically modified animal model as described herein.

In another aspect of the invention, genetically modified animal models for lymphopenia and related disorders are provided. In typical embodiments, the animal models exhibit one or more symptoms of type 1 diabetes (e.g., insulitis, abnormal blood glucose or insulin levels, etc.). Such an animal can be initially produced by promoting homologous recombination between an Ian5 gene in its chromosome and an exogenous, mutant Ian5 gene. The mutant Ian5 gene can be a null, hypermorph, neomorph, or hypomorph allele. In particular embodiments, the mutant Ian5 encodes a truncated mutant Ian5 polypeptide. For example, in a specific embodiment, the encoded truncated mutant Ian5 polypeptide is one having amino acids corresponding to positions 1-84 of SEQ ID NO: 3 and, optionally, 1-20 carboxy-terminal amino acids. In other embodiments, for production of “knockout animals,” the mutant Ian5 nucleic acid is rendered biologically inactive by, e.g., insertion of a heterologous sequence, such as an antibiotic resistance gene.

Methods for producing the genetically modified non-human animals are generally known in the art. For example, homologous recombination can be carried out by transforming embryo-derived stem (ES) cells with a vector containing the mutant Ian5 nucleic acid, such that homologous recombination occurs, followed by injecting the ES cells into a blastocyst, and implanting the blastocyst into a foster mother, followed by the birth of the chimeric animal in which the Ian5 gene has been modified (see, e.g., Capecchi, Science 244:1288-92 (1989); U.S. Pat. No. 6,204,061). The chimeric animal can be bred to produce additional genetically modified animals. Methods for producing genetically modified non-human animals are also disclosed in, e.g., U.S. Pat. No. 6,271,436. Such animals can be mice, rats, hamsters, sheep, pigs, cattle, and the like, and are typically non-human mammals. In a specific embodiment, a transgenic mouse is produced. In other embodiments, the animal is “humanized” to express the human Ian5 gene locus (and/or a corresponding human mutant Ian5 gene) using, e.g., the methods described above.

The genetically modified animals are expected to develop, or be predisposed to developing, lymphopenia and/or a related disorder. In a preferred embodiment, the animal model exhibits one or more clinical symptoms of type 1 diabetes. The animals are useful to screen for or test candidate compounds (e.g., Ian5 polynucleotides) for the ability to treat or prevent such disorders.

In another aspect, the present invention provides methods for developing gene therapy for the treatment of lymphopenia and/or related disorders. In a preferred embodiment, methods are provided for testing candidate agents for the treatment of type 1 diabetes. The methods can be used in non-human animals to test Ian5 polynucleotides (e.g., encoding wild-type Ian5 polypeptides or truncated mutant antisense) for amelioration of one or more symptoms of lymphopenia or a related disorder to identify, e.g., constructs, treatment regimes, etc. for further drug development. In certain embodiments of the method, the Ian5 polynucleotides are tested in genetically modified animal models for lymphopenia or a related disorder (see supra). In other embodiments, non-human animals known to have a mutation in the Iddml/lyp locus and which manifest lymphopenia and or other symptoms (e.g., DR.lyp/lyp rats having the rIan5(lyp) gene).

The method can be used to screen, for example, various Ian5 polynucleotide agents, including dosages, methods of delivery, target tissues, and the like. Animals are monitored before and following administration of the agents to determine treatment regimens that produce an amelioration of disease symptoms. Examples of Ian5 polynucleotides, vectors, delivery modes, target cells, etc., are further described herein.

Ian5 polynucleotides for testing in the animal models can be constructed using methods commonly known in the art of recombinant DNA technology (see, e.g., Ausubel et al, supra; Kriegler, Gene Transfer and Expression: A Laboratory Manual, Stockton Press, NY (1990)). In one embodiment, the agent comprises an Ian5 sense nucleic acid that is part of an expression vector that expresses an Ian5 polypeptide or fragment or chimeric protein thereof in a suitable host cell. In particular, such a nucleic acid has a promoter operatively linked to the Ian5 coding region, the promoter being inducible or constitutive, and, optionally, tissue-specific. Alternatively, the agent comprises an Ian5 antisense nucleic acid that is part of an expression vector that expresses the antisense nucleic acid in a suitable host. In particular, such an antisense nucleic acid has a promoter operatively linked to the Ian5 antisense nucleic acid, the promoter being inducible or constitutive, and, optionally, tissue-specific. In still other embodiments, the agent is an Ian5 antisense nucleic acid (such as, e.g., an antisense oligonucleiotide) that is not part of an expression vector.

In another particular embodiment, a nucleic acid is used in which the Ian5 coding sequences and any other desired sequences are flanked by regions that promote homologous recombination at a desired site in the genome, thus providing for intrachromosomal expression of the Ian5 nucleic acid (see, e.g., Koller and Smithies, Proc. Natl. Acad. Sci. USA 86:8932-35 (1989); Zijlstra et al., Nature 342:435-38 (1989); U.S. Pat. Nos. 5,631,153; 5,627,059; 5,487,992; and 5,464,764)). For example, wild-type Ian5 nucleic acids can be designed to integrate in the mutant Ian5 gene locus by homologous recombination.

For any of these embodiments, delivery of the nucleic acid into the non-human animal can be either direct (i.e., in vivo), in which case the animal is directly exposed to the nucleic acid or nucleic acid-carrying vector, or indirect (i.e., ex vivo), in which case cells are first transformed with the nucleic acid in vitro, then transplanted into the animal. In a specific embodiment, the nucleic acid is directly administered in vivo, where it is expressed to produce the encoded product. This can be accomplished by any of numerous methods known in the art (e.g., by constructing it as part of an appropriate nucleic acid expression vector and administering it so that it becomes intracellular, for example, by infection using a defective or attenuated retroviral or other viral vector (see, e.g., U.S. Pat. No. 4,980,286), by direct injection of naked DNA, or by use of microparticle bombardment, such as a gene gun (BIOLISTICTM, Dupont). DNA can also be inserted into cells by coating naked DNA with lipids or cell-surface receptors or transfecting agents, encapsulation in liposomes, microparticles, or microcapsules, or by administering the DNA in linkage to a peptide which is known to enter the nucleus, by administering the DNA in linkage to a ligand subject to receptor-mediated endocytosis (see, e.g., Wu and Wu, J. Biol. Chem. 262:4429-32 (1987)), which can be used to target cell types specifically expressing the receptors, and the like. In another embodiment, a nucleic acid-ligand complex can be formed in which the ligand comprises a fusogenic viral peptide to disrupt endosomes, allowing the nucleic acid to avoid lysosomal degradation.

In yet another embodiment, the nucleic acid can be targeted in vivo for cell specific uptake and expression by targeting a specific receptor (see, e.g., International Patent Publications WO 92/06180; WO 92/22635; WO 92/20316; WO 93/14188; and WO 93/20221). Alternatively, the nucleic acid can be introduced intracellularly and incorporated within host cell DNA for expression by homologous recombination (see, e.g., Koller and Smithies supra; Zijlstra et al. supra; U.S. Pat. Nos. 5,631,153; 5,627,059; 5,487,992; and 5,464,764).

Viral vectors that can be used include, e.g., retroviral vectors (see, e.g., Miller et al., Meth. Enzymol. 217:581-99 (1993); Boesen et al. (Biotherapy 6:291-302 (1994)) and lentiviral vectors (see, e.g., Naldini et al., Science 272:263-67 (1996)). Other vectors include, e.g., adenoviruses, which also are capable of infecting non-dividing cells (see, e.g., Kozarsky and Wilson, Curr. Opin. Genet Dev. 3:499-503 (1993); Bout et al. (Human Gene Therapy 5:3-10 (1994)) and adeno-associated virus (AAV) (see, e.g., Ali et al., Gene Therapy 1:367-84 (1994); U.S. Pat. Nos. 4,797,368 and 5,139,941; Walsh et al., Proc. Soc. Exp. Biol. Med. 204:289-300 (1993); Grimm et al., Human Gene Therapy 10:2445-50 (1999)).

A gene can be transferred to cells in tissue culture by methods such as electroporation, lipofection, calcium phosphate mediated transfection, or viral infection. Typically, the method of transfer includes the transfer of a selectable marker to the cells. The cells are then placed under selection to isolate those cells that have taken up and are expressing the transferred gene. The selected cells are then delivered to the non-human animal.

The nucleic acid can also be introduced into a cell prior to administration in vivo of the resulting recombinant cell. Such introduction can be carried out by any method known in the art, including, e.g., transfection, electroporation, microinjection, infection with a viral or bacteriophage vector containing the nucleic acid, cell fusion, chromosome-mediated gene transfer, microcell-mediated gene transfer, and the like. Numerous techniques are known in the art for the introduction of foreign genes into cells (see, e.g., Loeffler and Behr, Meth. Enzymol. 217:599-618 (1993); Cotten et al., Meth. Enzymol. 217:618-44 (1993); Cline, Pharmacol. Ther. 29:69-92 (1985)) and can be used in accordance with the methods of the present invention.

The resulting recombinant cells can be delivered to the non-human animal by various methods known in the art such as, for example, subcutaneous injection, application as a skin graft, intravenously administration (typically in the case of recombinant hemotopoietic cells such as, e.g., lymphocytes), etc.

Cells into which an Ian5 nucleic acid can be introduced for purposes of gene therapy encompass any desired, available cell type. For example, Ian5 can be introduced into hematopoietic cells such as, e.g., thymocytes, peripheral T lymphocytes, B lymphocytes, monocytes, macrophages, or various hematopoietic stem or progenitor cells (such as those obtained from bone marrow, umbilical cord blood, peripheral blood, fetal liver, and the like). The cells used for gene therapy are generally syngeneic to the animal (i.e., autologous or a genetically identical animal), but heterologous cells that can be typed for compatibility with the animal can be used.

In yet another aspect of the invention, methods for identifying in humans Ian5 mutations that correlate with type 1 diabetes or a related disorder are provided. In certain embodiments, the methods include determining the sequence of the Ian5 gene from a group of humans known to have type 1 diabetes and/or a related disorder and comparing these sequences to the wild-type human Ian5 gene sequence. Ian5 gene sequences from a control group of humans (e.g., not having type 1 diabetes and/or a related disorder as well as no family history of such disorders) can also be determined and compared to control for non-disorder related polymorphisms.

In a related aspect, methods for detecting in a subject the presence or risk of developing type I diabetes or a related disorder are provided. Biological samples containing polynucleotides are isolated from a subject and analyzed for mutations in the Ian5 gene locus. Mutations analyzed can be those resulting in a null, hypomorph, neomorph, or hypermorph allele. In certain embodiments, the mutation is a frameshift mutation resulting in a truncated mutant Ian5 polypeptide. In a particular embodiment, the frameshift mutation is in codon 85 of the human Ian5 coding sequence (i.e., in a codon corresponding to codon 84 of the rat Ian5 coding sequence). In one embodiment, the mutation is a deletion of one nucleotide at the third base pair position of codon 85. Detection of the mutation in the Ian5 gene indicates the presence or risk of developing type 1 diabetes or a related disorder.

There are many methods known in the art for detecting mutations in a given gene. Useful techniques include, for example, cloning and sequencing, ligation of oligonucleotides, use of the polymerase chain reaction and variations thereof (e.g., a PCR that uses 7-deaza-GTP), use of single nucleotide primer-guided extension assays, hybridization techniques using target-specific oligonucleotides that can be shown to preferentially bind to complementary sequences under given stringency conditions, sandwich hybridization methods, and the like.

Detection of mutations that correlate with type 1 diabetes can be performed, for example, by cloning and direct sequencing of the Ian5 gene sequences from subject samples. Sequencing may be carried out with commercially available automated sequencers utilizing labeled primers or terminators. An alternate sequencing strategy is sequencing by hybridization using high density oligonucleotide arrays on silicon chips (Fodor et al., Nature 364:555-556 (1993); Pease et al., Proc. Natl. Acad. Sci. USA 91:5022-5026 (1994)). Labeled target nucleic acid generated, for example, from PCR amplification of the target genes using fluorescently labeled primers, is hybridized with a chip containing a set of short oligonucleotides which probe regions of complementarity with the target sequence. The resulting hybridization patterns are used to reassemble the original target DNA sequence.

Oligonucleotide ligation involves methods based on ligation of oligonucleotide sequences which anneal immediately adjacent to each other on a target DNA or RNA molecule, including but not limited to the Ligase Chain Reaction or any other methods for the detection of specific mutations in nucleic acid sequences that are known to those skilled in the art (Wu and Wallace, Genomics 4:560-569 (1989); Landren et al., Science 241:1077-1080 (1988); Nickerson et al., Proc. Natl. Acad. Sci. 87:8923-8927 (1990); Barany, Proc. Natl. Acad. Sci. 88:189-193 (1991)). Ligase-mediated covalent attachment occurs only when the oligonucleotides are correctly base-paired. For example, one useful method is the Ligase Chain Reaction (LCR), which utilizes the thermostable Taq ligase for target amplification. The elevated reaction temperatures permits the ligation reaction to be conducted with high stringency (Barany, PCR Methods and Applications 1:5-16 (1991)).

In addition, analysis of point mutations in DNA may be carried out using polymerase chain reaction (PCR) and variations thereof (e.g., using 7-deaza GTP with or instead of dGTP). Mismatches are detected by competitive oligonucleotide priming under hybridization conditions where binding of the perfectly matched primer is favored (Gibbs et al., Nucl. Acids Res. 17:2437-2448 (1989)). In the amplification refractory mutation system technique (ARMS), primers are designed to have perfect matches or mismatches with target sequences either internal or at the 3′ residue (Newton et al., Nucl. Acids Res. 17:2503-2516 (1989)). Under appropriate conditions, only the perfectly annealed oligonucleotide functions as a primer for the PCR reaction, thus providing a method of discrimination between normal and mutant Ian5 sequences.

Genotyping analysis of Ian5 sequences may also be carried out using single nucleotide primer-guided extension assays, where the specific incorporation of the correct base is provided by the high fidelity of the DNA polymerase (See, e.g., Syvanen et al., Genomics 8:684-592 (1990); Kuppuswamy et al., Proc. Natl. Acad. Sci. USA 88:1143-1147 (1991)).

Differential hybridization techniques using target-specific oligonucleotides can be used to detect single base mutations in target nucleic acids. (See, e.g., Suggs et al., Proc. Natl. Acad. Sci. USA 78:6613-6617 (1981); Conner et al., Proc. Natl. Acad. Sci. USA 80:278-282 (1983); Saiki et al., Proc. Natl. Acad. Sci. USA 86:6230-6234 (1989).) Mutations are diagnosed on the basis of the higher thermal stability of the perfectly matched probes as compared to the mismatched probes. The hybridization reactions are carried out in a filter-based formal, in which the target nucleic acids are immobilized on nitrocellulose or nylon membranes and probed with oligonucleotide probes. Any of the known hybridization formats may be used, including Southern blots, slot blots, “reverse” dot blots, solution hybridization, solid support based sandwich hybridization, and bead-based, silicon chip-based, and microtiter well-based hybridization formats.

An alternative strategy involves detection of Ian5 nucleic acid sequences by sandwich hybridization methods. In this strategy, the mutant and wildtype target nucleic acids are separated from non-homologous DNA/RNA using a common capture oligonucleotide immobilized on a solid support and detected by specific oligonucleotide probes tagged with reporter labels. The captured oligonucleotides are immobilized on microtiter plate wells or on beads (Gingeras et al., J. Infect. Dis. 164:1066-1074 (1991): Richman et al., Proc. Natl. Acad. Sci. USA 88:11241-11245 (1991)). Radio-isotopic or non-isotopic labeled detection oligonucleotide probes can be used. A number of strategies are available for detecting target nucleic acids by non-isotopic means (Matthews et al., Anal. Biochem. 169:1-25 (1988)). The non-isotopic detection method may be either direct or indirect.

In an indirect detection process, the oligonucleotide probe is generally covalently labeled with a hapten or ligand such as digoxigenin or biotin. Following the hybridization step, the target-probe duplex may be detected by an antibody- or streptavidin-enzyme complex. Enzymes commonly used in DNA diagnostics are horseradish peroxidase and alkaline phosphatase. For example, one indirect method, the Genius detection system (Boehringer Mannheim), uses digoxigenin as the tag for the oligonucleotide probe and is detected by an anti-digoxigenin-antibody-alkaline phosphatase conjugate.

Direct detection methods include the use of fluorophore-labeled oligonucleotides, lanthanide chelate-labeled oligonucleotides or oligonucleotide-enzyme conjugates. Examples of fluorophore labels are fluorescein, rhodamine and phthalocyanine dyes. Examples of lanthanide chelates include complexes of Eu³⁺ and Tb³⁺. Directly labeled oligonucleotide-enzyme conjugates are preferred for detecting point mutations when using target-specific oligonucleotides, as they provide very high sensitivities of detection. Oligonucleotide-enzyme conjugates are prepared by a number of methods (see, e.g., Jablonski et al., Nucl. Acids Res. 14:6115-6128 (1986); Li et al., Nucl. Acids Res. 15:5275-5287 (1987); Ghosh et al., Bioconjugate Chem. 1:71-76 (1990)), with alkaline phosphatase typically used for obtaining high sensitivities of detection. The detection of target nucleic acids using these conjugates may be carried out by filter hybridization methods or by bead-based sandwich hybridization (see, e.g., Ishii et al., Bioconjugate Chemistry 4:34-41 (1993)).

Detection of the probe label may be accomplished using the following approaches. For radioisotopes, detection may be by autoradiography, scintillation counting, or phosphor imaging. For hapten or biotin labels, probe may be detected by antibody or streptavidin bound to a reporter enzyme such as horseradish peroxidase or alkaline phosphatase, which is then detected by enzymatic means. For fluorophore or lanthanide-chelate labels, fluorescent signals may be measured with spectrofluorimeters with or without time-resolved mode or using automated microtiter plate readers. With enzyme labels, detection may be by color or dye deposition (p-nitrophenyl phosphate or 5-bromo-4-chloro-3-indolyl phosphate/nitroblue tetrazolium for alkaline phosphatase and 3,3′-diaminobenzidine-NiCl₂ for horseradish peroxidase), fluorescence (e.g., 4-methyl umbelliferyl phosphate for alkaline phosphatase) or chemiluminescence (the alkaline phosphatase dioxetane substrates LumiPhos 530 from Lumigen Inc., Detroit Mich. or AMPPD and CSPD from Tropix, Inc.). Chemiluminescent detection may be carried out with X-ray or Polaroid film or by using single photon counting luminometers, which is the typical detection format for alkaline phosphatase labeled probes.

The detection oligonucleotide probes range in size between 10-100 bases, and are preferably between 15 to 30 bases in length. In order to obtain the required target discrimination using the detection oligonucleotide probes, the hybridization reactions are generally run between 20°-60° C., and most preferably between 30°-50° C. As known to those skilled in the art, optimal discrimination between perfect and mismatched duplexes is obtained by manipulating the temperature and/or salt concentrations or inclusion of formamide in the stringency washes.

Measuring Ian5 Expression for Detection of Presence or Risk of Diabetes

In another aspect, as an alternative to detection of mutations in Ian5 nucleic acids associated with type 1 diabetes or a related disorder, the method for detecting the presence or risk of type 1 diabetes or a related disorder comprises detecting the level of Ian5 gene expression in a subject and/or analyzing Ian5 polypeptides. Biological samples are isolated from a subject for analysis of gene expression using, e.g., nucleic acid probe hybridization analysis or immune techniques. Biological samples are generally selected based on normal Ian5 expression patterns. Thus, typical samples can include, e.g., peripheral blood lymphocytes. Reduced expression of normal Ian5 polypeptide and/or the presence of abnormal Ian5 polypeptides (such as, e.g., a truncated mutant Ian5) is indicative of the presence of or risk of developing type 1 diabetes or a related disorder.

In certain embodiments, Ian5 gene expression may be analyzed using nucleic acid probes (e.g., directly or indirectly labeled, see supra). Methods for detection of mRNA transcripts using hybridization analysis are generally known in the art. (See, e.g., Sambrook et al.; Ausubel et al.) Hybridization of the probe can be in vitro (e.g., Northern blot analysis, RNAse protections assays, and the like) or in situ (e.g., fluorescent in situ hybridization; see, e.g., In Situ Hybridization: A Practical Approach (IRL Press, D. G. Wilkinson ed., 1994)).

In other embodiments, Ian5 polypeptides can be analyzed using anti-Ian5 specific antibodies (see, e.g., supra). Antibodies can be generated which are specific for normal Ian5 polypeptide and used in standard immunoassays (e.g., in situ immunohistochemistry, Western blot analysis, FACS analysis, and the like). In certain embodiments, antibodies are raised against particular regions or domains of an Ian5 polypeptide, against conformational epitopes of Ian5, or that are specific for wild-type or mutant (e.g., truncated mutant) forms of Ian5. Antibodies specific for particular domains, conformational epitopes, or isoforms of Ian5 can also be used to further analyze the Ian5 polypeptides expressed in a sample (e.g., to determine the presence of mutant Ian5 polypeptides). For example, sample binding of antibodies against particular domains or conformational epitopes can be compared, e.g., with sample binding of antibodies that are non-domain specific (e.g., polyclonal or a cocktail of monoclonal antibodies) or against non-conformational epitopes, respectively, to determine the presence of Ian5 polypeptides which lack normal domain or conformational structure (such as, e.g., truncated mutant Ian5 polypeptides).

A further understanding of the present invention will be obtained by reference to the following description that sets forth illustrative embodiments.

### EXAMPLE 1

### Prediction of Diabetes by E517P-GAD65 Mutation

GAD65 Antibodies

Three types of GAD65-specific antibody reagents were used: group A:monoclonal antibodies; group B: polyclonal antibodies and group C: standard patient sera (Table 1). In group A, mouse MAbl44 was used that binds to the N-terminus of GAD65 (amino acid residues 4-22) and was developed in our laboratory (Hampe et al., J. Neuroimmunology 113:63-71 (2001)), as well as mouse monoclonal antibody GAD6 that recognizes the C-terminus of GAD65 (Ujihara et al., Diabetes 43:968-975 (1994), Richter et al., Proc. Natl. Acad. Sci. USA 90:2832-2836 (1993), Chang et al., J. Neurosci. 8:2123-2130 (1988)). The isolation of human MAbs, MICA-1, MICA-2, MICA-3, MICA-4, and MICA-6 was previously described (Richter et al., Proc. Natl. Acad. Sci. USA 89:846-847 (1992), Richter et al., Proc. Natl. Acad. Sci. USA 90:2832-2836 (1993)). In group B, rabbit antisera were previously raised by immunization with synthetic peptides corresponding to peptide sequences specific for human GAD65 molecule (Li et al., J. Histochem. and Cytochem. 43:53-59 (1995)). The antiserum #6952 and #7309 were against an epitope at amino acid position 4-22 in GAD65; #7222 with amino acids 73-91; #8100 and #7641 with amino acids 250-269; #5545, #5551, #5581, #5576 with amino acids 390-403; #5482, #5587 with amino acids 405-418; and #7646 with amino acids 570-585. In group C, ten well studied standard sera were from 11-16 years of age newly diagnosed type 1 diabetes children treated with plasma-pheresis at clinical onset (Baekkeskov et al., Nature 298:167-169 (1982), Ludvigsson et al., Br. Med. J 286:176-178 (1983), Marner et al., Diabetes Res. 2:231-236 (1985), Rabinovitch et al., Diabetes 33:224-228 (1984)).

TABLE 1
GAD65 antibody reagents used.

| Group | Antibody | Species | Antigen | Reference** |
|---|---|---|---|---|
| A | Mab144 | mouse | 4-22 GAD65 peptide | A |
| | GAD6 | mouse | purified GAD65 | C |
| | MICA1 | human | patient Mab | B, D |
| | MICA2 | human | patient Mab | B, D |
| | MICA3 | human | patient Mab | B, D |
| | MICA4 | human | patient Mab | B, D |
| | MICA6 | human | patient Mab | B, D |
| B | #6952 | rabbit | 4-22 GAD65 peptide | E |
| | #7309 | rabbit | 4-22 | E |
| | #7222 | rabbit | 73-91 | E |
| | #7641 | rabbit | 250-269 | E |
| | #8100 | rabbit | 250-269 | E |
| | #5545 | rabbit | 390-403 | E |
| | #5050 | rabbit | 390-403 | E |
| | #5551 | rabbit | 390-403 | E |
| | #5576 | rabbit | 390-403 | E |
| | #5581 | rabbit | 390-403 | E |
| | #5482 | rabbit | 405-418 | E |
| | #5547 | rabbit | 405-418 | E |
| | #5571 | rabbit | 405-418 | E |
| | #5587 | rabbit | 405-418 | E |
| | #7647 | rabbit | 570-585 | E |
| C | 591 | Type 1 | Female, 13 years, | F |
| | | patient | DR3/4 | |
| | (1) | | Male, 12, DR3/4 | F |
| | 622 | | Male, 15, DR3/4 | F |
| | 652 | | Male, 14, DR3/4 | F |
| | 673* | | Male, 11, DR3/4 | G |
| | 686 | | Male, 10, DR3/4 | F |
| | 708 | | Male, 16, DR3/4 | F |
| | 826 | | Male, 12, DR3/4 | F |
| | 853 | | Male, 12, DR3 | F |
| | 898 | | Male, 13, DR3/4 | F |

*This serum is the WHO standard for ICA and GAD65 Ab.
**A = Hampe et al., J. Neuroimmunology 113: 63-71 (2001)
B = Richter et al., Proc. Natl. Acad. Sci. USA 90: 2832-2836 (1993)
C = Chang et al., J. Neurosci. 8: 2123-2130 (1988)
D = Richter et al., Proc. Natl. Acad. Sci. USA 89: 846-847 (1992)
E = Li et al., J. Histochem. and Cytochem. 43: 53-59 (1995)
F = Ludvigsson et al., Br. Med. J. 286: 176-178 (1983)
G = Mire-Sluis et al., Diabetes Metab. Res. Rev. 15: 72-77 (1999)

Patients and Control Subjects

Five groups of GAD65Ab positive human serum samples were used in this study. The serum samples were all from previously reported GAD65Ab positive subjects (Baekkeskov et al., Nature 298:167-169 (1982), Ludvigsson et al., Br. Med. J. 286:176-178 (1983)). Type 1 diabetes patients (n=95) and Type 2 patients (n=28), who registered in 1992-1993 in the Diabetes Incidence Study in Sweden (Baekkeskov et al., Nature 298:167-169 (1982), Ludvigsson et al., Br. Med. J. 286:176-178 (1983)), were included along with first degree relatives (n=36) and SMS patients (n=5). In addition, GAD65Ab positive healthy subjects (n=54) identified following the screening of 2800 population based subjects (Schranz et al., Journal of Immunological Methods 213:87-97 (1998)) were also studied.

Polymerase Chain Reaction-Mediated Mutagenesis

For PCR amplification, 1 pg of plasmid pEx9 DNA (Grubin et al., Diabetologia 37:344-350 (1994)) was used. The volume was adjusted to 50 μl by adding 35 μl H₂O, 50 μl PCR buffer (10× concentrated; 500 mM KCl, 100 mM Tris pH 8.3), 4 μl 25 mM MgCl₂, 0.5 μl each of two primers (15 μM), 1 μl each of dATP, dCTP, dGTP, and dTTP (2.5 mM) and 0.5 unit Pfu DNA polymerase (Stratagene, La Jolla, Calif.). The PCR amplification was carried out for 30 cycles in a Thermal Cycler (PTC-200, MJ Research, Watertown, Mass.). Each cycle included denaturation at 94° C. for 1 min, reannealing of primers at 55° C. for 1 min and extension at 72° C. for 1 min. The PCR products (10 μl) were applied onto 1% (v/v) agarose gels for analysis. The sequence structures for one of the single amino acid substitutions (Table 2) of the mutagenic primers (mutations underlined) used for PCR are listed below.

| | | |
|---|---|---|
| DL-1: | 5′-GTATAAGATCTGGATGCATG-3′ (BglII) | (SEQ ID NO: 8) |
| DL-2: | 5′-GCCCTCTAGAGAAGTGGAACAG-3′ (XbaI) | (SEQ ID NO: 9) |
| DL-3: | 5′-GCGTACTCTGCCAGACAATGAAG-3′ | (SEQ ID NO: 10) |
| DL-4: | 5′-CTTCATTGTCTGGCAGAGTACGC-3′ | (SEQ ID NO: 11) |

Plasmid pEx9-GAD-E517P was generated by replacing BglII/XbaI fragments of pEx9 with PCR-amplified DNA products which already had base-substitutions by the PCR-mediated mutagenesis. All subcloned DNA fragments used in this study and listed in Table 2 were sequenced for verification of the nucleotide point mutations.

In Vitro Transcription/Translation and Radioimmunoassay

In vitro transcription-translation reactions with SP6 RNA polymerase and rabbit reticulocyte lysate (Promega, Madison, Wis.) were performed following the manufacturer's instructions to prepare ³⁵S-labeled GAD65 and its variant molecules (Grubin et al., Diabetologia 37:344-350 (1994)).

The GAD65 binding activity was determined by Protein A-Sepharose mediated radioimmunoassay (RIA), as previously described (Hao et al., Diabetes Technology & Therapeutics 1: 13-20 (1999), Schranz et al., J. Immunol. Methods 213:87-97 (1998), Hampe et al., J. Clin. Endocrinol. Metab. 84:643-648 (1999)). Briefly, ³⁵S-labeled protein (20,000 cpm of TCA precipitable radioactivity) was incubated with antibody preparations at different dilutions and incubated overnight at 4° C. Antibody-bound and free antigen was separated by 40% (v/v) protein A-Sepharose in Millipore multiscreen plates (MABVNOB50, Millipore, Bedford, Mass.). Radioactivity in the plates was counted in a Wallac 1450 Micro Beta Scintillation Counter (Wallac, Turku, Finland).

Results

Generation of C-Terminal GAD65/GAD67 Chimeric Constructs

Amino acid sequence comparison between GAD65 and GAD67 at the C-terminal region (AA position 439-585) revealed a distinct feature with marked sequence dissimilarity at amino acid positions 515-525, whereas the remaining part of C-terminal region shared about 82% sequence identity. The 515-525 region has previously not been implicated in antibody binding (Schwartz et al., J. Mol. Biol. 287:983-999 (1999)), but was suggested as a T cell epitope for GAD65 in human (Patel et al., Proc. Natl. Acad. Sci. USA 94:8082-8087 (1997)), as well as in the NOD mouse (Chao et al., Immunogenetics 46:29-34 (1997)). The hypothesis that amino acid residues in the 515-525 region contributed to GAD65Ab conformational epitope was therefore tested. A panel of four GAD65/GAD67 chimeras were generated to cover this small region of 11 amino acid residuals (Table 2). The resulting GAD65/GAD67 chimeric proteins were expressed by in vitro coupled transcription translation. The immunoreactivity of the ³⁵S-labeled molecules was analyzed by RIA to determine antigen-antibody binding. The chimeric constructs were tested with the Group A-C sera summarized in Table 1. The monoclonal GAD65 antibodies MAb144 and GAD6, as well as MICAs (group A) were followed by polyclonal rabbit sera to GAD65Ab epitopes (Group B) and by a group of sera from ten newly diagnosed type 1 diabetes patients (group C). This group of sera has been used to identify islet cell autoantigens and to standardize their autoantibodies (Baekkeskov et al., Nature 298:167-169 (1982), Ludvigsson et al., Br. Med. J. 286-176-178 (1983), Marner et al., Diabetes 2:231-236 (1985), Rabinovitch et al., Diabetes 33:224-228 (1984)), including the 97/550 WHO standard for islet cell antibodies and GAD65Ab (Mire-Sluis et al., Diabetes Metab. Res. Rev. 15:72-77 (1999)). Compared to wild-type GAD65 and in contrast one chimeric construct (GAD65-REK), a marked loss was observed of the binding activity to GAD65-GVP in group A and C antibodies (Table 1). However, further mutations down streams of position GVP in two chimeric molecules (GAD65-GVP-SPQ and GAD65-GVP-SPQ-REK) completely recovered the loss of antibody binding activity. These results indicated that GAD65-specificsequences required for binding to a conformation dependent epitope was determined by the GVP substitutions within the C-terminal region of the GAD65 molecule.

TABLE 2

Glutamic acid decarboxylase (GAD) constructs
and antibody reactivity

| | |
|---|---|
| | Binding with |
| GAD65 515-525 | antibodies |

| | | | |
|---|---|---|---|
| Name | sequences | Group A′ | Group C |

| | | | |
|---|---|---|---|
| GAD65 | TLEDNEERMSR | + | + |
| | (SEQ. ID No:12) | | |
| GAD67 | GVPDSPQRREK | − | (+) |
| | (SEQ. ID No:13) | | |
| GAD65-REK | TLEDNEERREK | + | + |
| | (SEQ. ID No:14) | | |
| GAD65-GVP | GVPDNEERMSR | − | − |
| | (SEQ. ID No:15) | | |
| GAD65-E517P | TLPDNEERMSR | − | − |
| | (SEQ. ID No:16) | | |
| GAD65-E517A | TLADNEERMSR | + | + |
| | (SEQ. ID No:17) | | |
| GAD65-GVP-SPQ | GVPDSPQRMSR | + | + |
| | (SEQ. ID No:18) | | |
| GAD65-GVP-SPQ- | GVPDSPQRREK | + | + |
| REK | (SEQ. ID No:19) | | |

¹Excluding Mab 144 antibody.

Site-Directed Mutagenesis of E517P

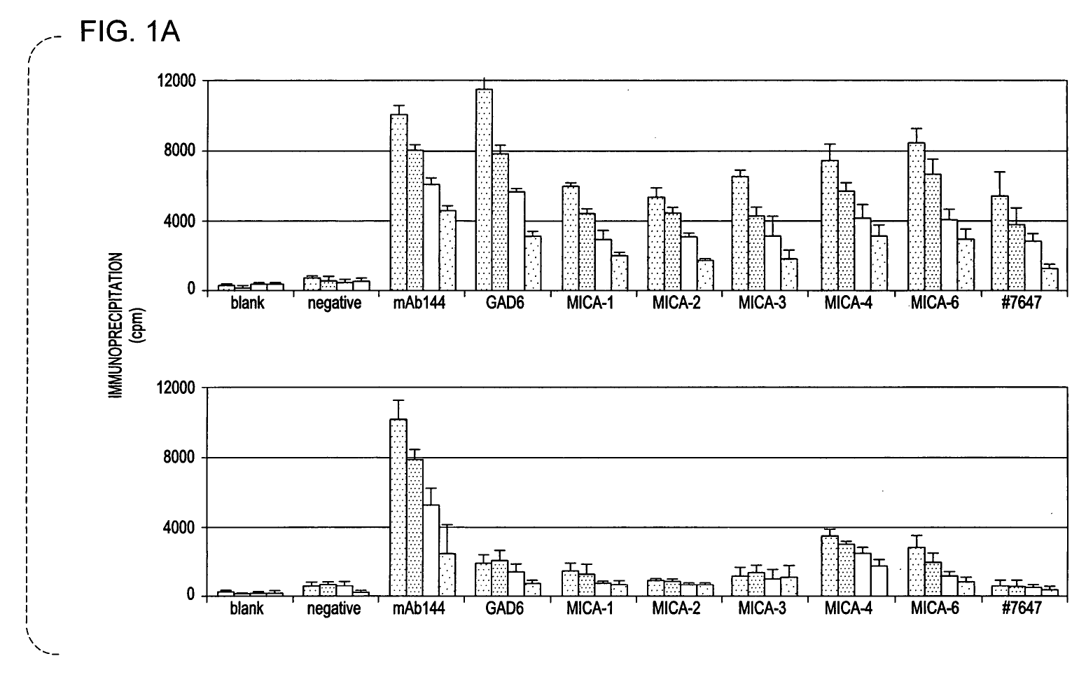

To localize a specific amino acid residue within the epitope sequence that was responsible for the loss of antibody binding, a point mutation was created by substituting Glu by Pro (the corresponding GAD67 residue) at position of 517 in GAD65. The point mutation (E517P) was established by PCR-mediated mutagenesis and verified by nucleotide sequence analysis. The transcription translation efficiency of E517P-GAD65 did not differ from wild-type GAD65. To analyze E517P-targeted epitopes and define possible interactive sequences in other GAD65 regions, the binding of rabbit polyclonal antibodies (group B antibodies) that recognize different sequence segments in GAD65 molecule was analyzed. As shown in FIG. 1B, the E517P point mutation affected antibody binding mostly in C-terminal and middle regions, but not at the N-terminal region and at the GAD65 enzymatic active site. Within the middle region, stronger reduction of antibody binding by E517P was observed immediately downstream rather than upstream of the enzyme active site (FIG. 1B). This result suggested that sequences in the middle and C-terminal regions interact to form a conformation important to GAD65Ab recognition. The E517P mutation may dictate this conformation dependent GAD65 epitope.

To support this observation, E517P-GAD65 was tested by group A antibodies, including MAb 144, GAD6, and MICA, that recognized different part of GAD65 molecule. As shown in FIG. 1A, the Mab144 antibody binding was not affected by the E517P-GAD65 mutation. In marked contrast, the E517P mutation affected the binding of GAD6 and MICA, with reductions ranging from 52% to 83% (FIG. 1A). Interestingly, only moderate reduction of the binding in E517P-GAD65 was observed by MICA-4 and MICA-6, which recognize sequences upstream of the enzymic active site (Schwartz et al., J. Mol. Biol. 287:983-999 (1999), Richter et al., Proc. Natl. Acad. Sci. USA 90:2832-2836 (1993)). To further analyze whether the loss of antibody binding in E517P-GAD65 was caused by individual amino acid substitution of Glu to Pro, or by a position effect at 517, the E517A-GAD65 mutation was created by exchanging Pro to Ala at position 517. Binding experiments using group A and C antibodies demonstrated same binding between E517A-GAD65 and wild-type GAD65. This suggested that the Pro residue rather than a position effect might be a key amino acid for the E517P-sensitive conformational GAD65 epitope.

Analysis of GAD65 Antibody Positive Human Sera

Experiments were performed to test whether antibody binding to the E517P-GAD65 mutant distinguished GAD65Ab in type 1 diabetes patients from GAD65Ab in subjects without immediate risk for the disease. First, the standard type 1 diabetes group C serum samples (Table 1) were analyzed. In spite of two GAD65Ab negative serum samples (#853 and #898), eight of ten samples exhibited dramatic decrease of binding to E517P-GAD65, with reductions ranging from 60% to 86% of GAD65Ab (100%) binding (FIG. 1C). This result demonstrated that GAD65 specific autoantibodies present at the onset of type 1 diabetes patients seemed heavily dependent on the epitope associated with the E517 position since the E517P mutation dramatically affected the GAD65Ab binding.

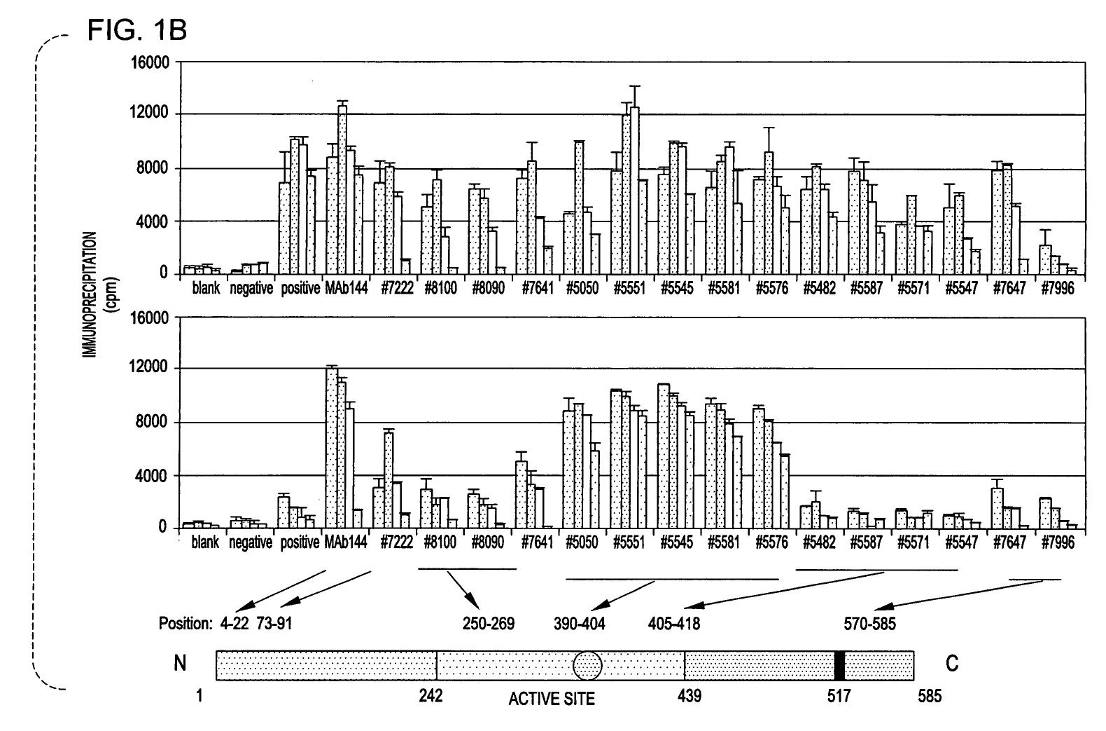

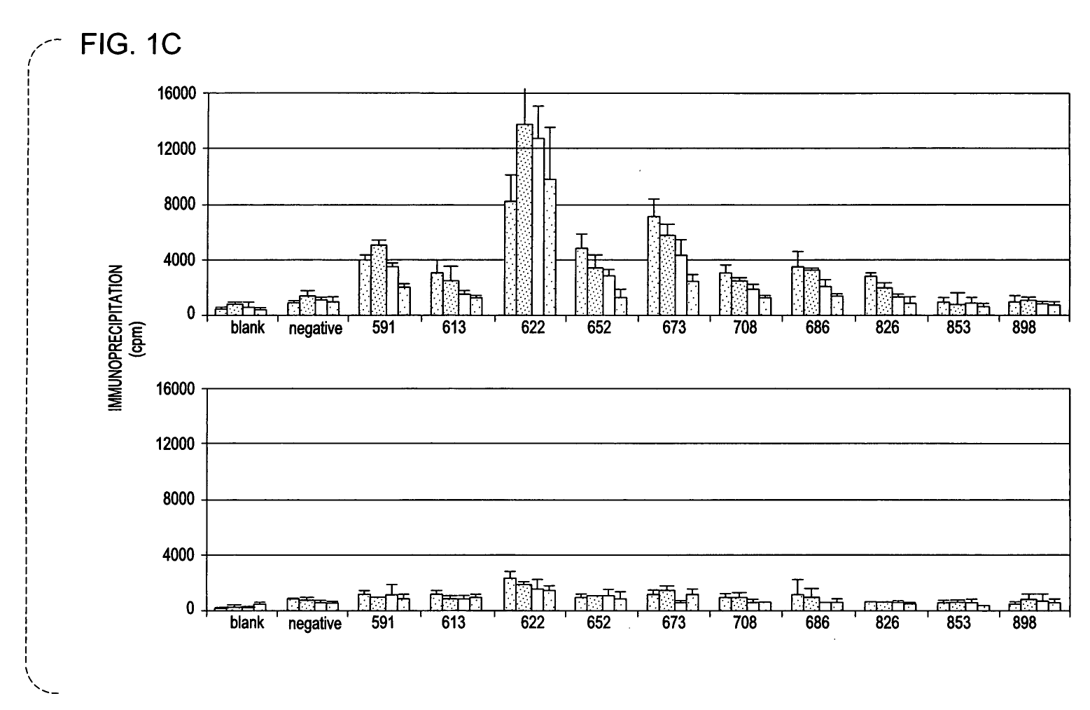

To further determine the significance of the E517P mutation, experiments were performed to analyze a total of 218 human serum samples that were divided into five groups: a) 95 new onset type 1 diabetes patients; b) 28 new onset GAD65Ab-positive type 2 diabetes patients; c) 36 healthy first degree relatives of siblings or parents to type 1 diabetes patients; d) 5 SMS patients, and e) 54 GAD65Ab positive healthy subjects identified by population based screening (Rolandsson et al., Diabetologia 42:555-559 (1999)). Autoantibody binding to E517P-GAD65 was compared to wild-type GAD65 at identical assay conditions. The binding distribution pattern was much different among the five groups (FIG. 2A). In healthy controls, most (50/54) of samples showed only moderated reduction, the mean binding being 72% of the binding to wild-type GAD65 (100%) (P<0.0001), only four samples exhibited a dramatic reduction (below 25% of GAD65Ab value, P<0.0001). In contrast to the healthy control group, a marked reduction in autoantibody binding to E517P-GAD65 was observed in type 1 diabetes sera; the mean binding was 33% of GAD65 binding. The reduction in binding to E517P-GAD65 was independent of the GAD65Ab titer. Out of 95 type 1 diabetes patients, 85 (89%) ranged in binding from 10% to 50% of the GAD65 binding, indicating that the majority of the patient sera were sensitive to the E517P mutation. However, ten patients were less affected by E517P and maintained binding to the mutant that was 65%-100% of GAD65 (FIG. 2A). Further analysis demonstrated that these ten patients were positive for both GAD65Ab and GAD67Ab whereas the remaining 85 patients were GAD65Ab positive but GAD67Ab negative (FIG. 3).

GAD65Ab positive type 2 diabetes patients showed a similar pattern to type 1 diabetes patients (FIG. 2A). As many as 24/28 (86%) patient sera showed binding to E517P-GAD65 that was less than 50% of that to GAD65 (mean 29%; range 5 to 100%). Again, four patient sera showed binding to E517P-GAD65 above 50% GAD65 were GAD65Ab/GAD67Ab double positive, suggesting the presence of autoantibodies to shared GAD65/67 epitopes that are insensitive to the E517P mutation. In the 36 GAD65Ab positive first degree relatives, the distribution in binding to the E517P-GAD65 mutant ranged from 1-100%. The mean binding was 44% which was not different from the 100% binding to GAD65 (P<0.0001). It was found that 7/36 serum samples from the first degree relatives were positive for both GAD65 and GAD67 autoantibodies (FIG. 3). First-degree relatives with GAD65Ab have a variable risk for disease (Bingley et al., Diabetes Care 22:1796-1801 (1999), Greenbaum et al., Diabetes 48:170-175 (1999)). Thus, this group of subjects showed a binding pattern to E517P-GAD65 that was intermediary between healthy subjects and type 1 diabetes patients. Finally, in support of this conclusion, 14% GAD65Ab positive health subjects were also positive for GAD67Ab. Double positivity did however only explain 4/54 (7%) of the sera that showed a reduction of binding to E517P-GAD65 that was less than 50%. Hence, there is more to the conformational sensitivity of type 1 diabetes sera than GAD65/67Ab double positivity.

Secondary Structural Prediction

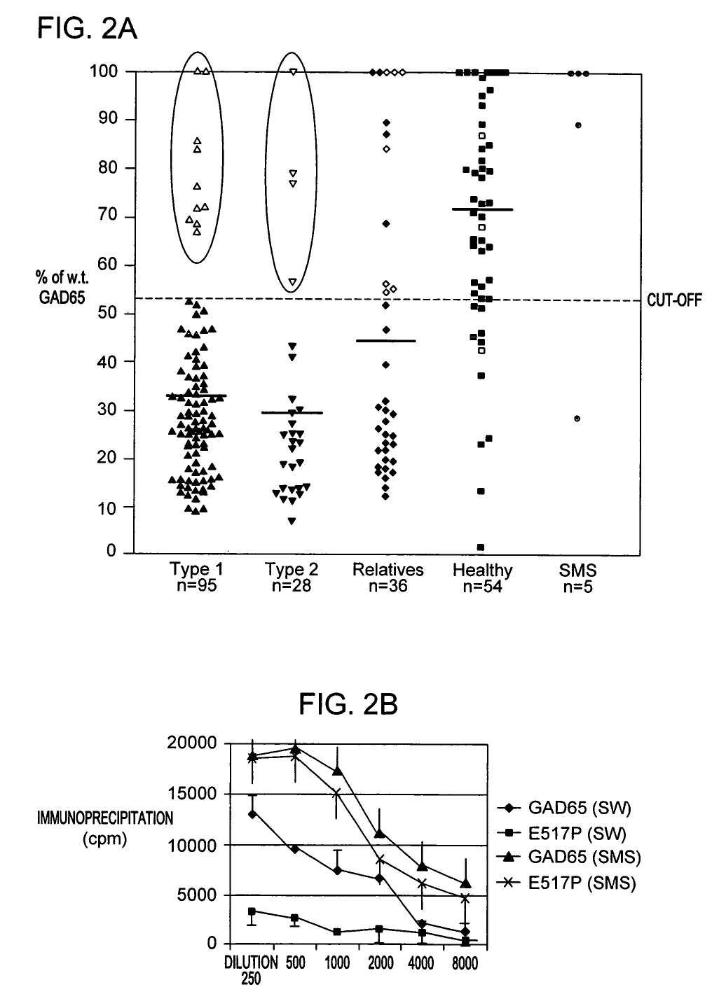

Secondary structural predictions at the E517 region of GAD65/67 indicated major effects by the E517P mutation. Short peptide sequences (from position 505 to 544) from GAD65, GAD67, and E517P-GAD65 were analyzed using the NCI computer modeling system. As illustrated in FIG. 4, both GAD65 and GAD67 peptide sequences formed an alpha-helix structure, but the helix positions were different. Three motifs: I, II, and III were discernible. Motif I was missing in GAD67. Motifs II and III remained intact in both GAD65 and GAD67. Interestingly, motif I was not formed in GAD65-GVP and E517P-GAD65, indicating a possible structurally mimicry between GAD65-GVP or E517P-GAD65 and GAD67. Such shift to GAD67-like structure at a local region by the E517P-GAD65 mutation may explain the markedly reduced binding of type 1 diabetes associated GAD65-specific autoantibodies.

### EXAMPLE 2

### Lymphopenia in the BB Rat Model of Type 1 Diabetes is Due to a Mutation in the Ian5 Gene

Methods

Rats

BB DR (Bieg et al, Mamm. Genome 9:324-326 (1998)) and F344 rats (Klaff et al., Mamm. Genome 10:883-887 (1999)) congenic for lymphopenia were maintained at the University of Washington. All animals were kept under specific pathogen free (SPF) conditions with standard light-dark cycles. The rats were fed a regular diet. Sentinel animals were negative for viral antibodies and parasites during the period of the experiments. Siblings heterozygous for polymorphic markers flanking the lymphopenia interval were used as breeding pairs to generate homozygous animals. The rats were screened for diabetes and lymphopenia as described in detail in Bieg et al., Mamm. Genome 9:324-326 (1998). DNA was obtained from 32 different rat strains as described (Kwitek et al., Genome Res. 11:1935-1943 (2001)). In addition, DNA from LEA, LEC, OLETF, and WKAH rats (obtained from Dr. Kozo Matsumoto, University of Tokushima, Japan) and from outbred BBDR and BBDP rats (obtained from Health Products & Food Branch, Sir Frederick Banting Research Centre, Ottawa, Ontario KIA OLZ Canada) was also analyzed.

Physical Mapping and STS Screening

Mouse YAC Contig

Mouse YAC contigs were generated by first screening with known STSs and then filling in gaps by sequencing YAC ends and using resulting non-repetitive sequence as additional STSs. Unless preexisting or otherwise noted, PCR primers were selected using the Primer 0.5 program (Lincoln et al., Primer: A Computer Program for Automatically Selecting PCR Primers, MIT Center for Genome Research and Whitehead Institute for Biomedical Research, (1991)) to choose primers with predicted melting temperatures within 1° C. of 60° C. and to avoid regions with repeat- or self-similarity. PCR amplification was performed according to the conditions specified for each protocol, or as previously. described, or, if not specified, according to standard conditions as recommended by Perkin-Elmer. YACs were isolated from the MIT mouse YAC library (Kusumi et al., Mamm. Genome 4:391-392 (1993)) using standard PCR screening methods and the YAC DNA prepared as described (Segre et al., Genomics 28:549-559 (1995)). YAC ends were isolated using inverse PCR as previously described (Haldi et al., Genomics 24: (1994)) and sequenced directly using standard fluorescent sequencing methods.

Higher Resolution Physical Mapping

Mouse bacteriophage P1 clones were isolated from two libraries, the P1 mouse RRII (2-3× coverage) and the P1 mouse ES (3× coverage) libraries (Pierce et al., Mamm. Genome 3:550-558 (1992); Stemberg et al., GATA 11:171-180 (1994)) (Genome Systems, St Louis, Mo.). Mouse BAC clones were isolated from a 129/SV mouse BAC library CITB CJ7B (7× coverage) (Kim et al., J. Immunol. 157:5461-5466 (1996); Shizuya et al., PNAS 89:8794-8797 (1992)). Rat PAC clones were isolated from the RPCI-31 library (Woon et al., Genomics 50:306-316 (1998)). Each library was screened by a PCR-based or hybrid PCR- and hybridization-based protocol, as recommended by the library maker. P1, BAC, and PAC DNA was prepared according to standard protocols and as recommended by Genome Systems. P1, BAC and PAC end sequences were obtained using a protocol similar to that for cloning YAC ends. STS content maps were assembled by using standard PCR techniques to determine the STS content of panels of miniprep DNA from the isolated PIs, BACs, and PACs.

Cross-Species cDNA Selection

“Cross-Species” cDNA Selection was performed using a modified protocol from that previously described by Lovett (Lovett et al., Biochem. Biophys. Res. Comm. 144:1069-1075 (1987) and Lovett et al., PNAS 88:9628-9632 (1991)) and the primers cDNA-1 (5′ CTGAGCGGAATTCGTG AGACC 3′) (SEQ ID NO: 20)/cDNA-2 (5′ P-GGTCTCACGAATTCCGCTCAGTT 3′) (SEQ ID NO: 21). All mouse template cDNAs were separately PCR amplified 10-15 cycles (94° C./64° C./72° C.) with the bio-cDNA 1 primer (5′ biotin-CTGAGCGGAATTCGTGAGACC 3′ (SEQ ID NO: 20); 64.4° C. predicted melting temperature) and purified. Double-stranded rat cDNA from testis and spleen with an average fragment size of approximately 500 bp was modified with linkers composed of the two oligos cDNA-lb (5′ GTCACGCAAGCTTCTC ACAGG 3′) (SEQ ID NO: 22) and cDNA-2b (5′ P-CCTGTGAGAAGCTTGCGTGACTT 3′) (SEQ ID NO: 23) and amplified using the cDNA-lb primer. One μg amplified cDNA, 2 μg mouse Cₒt-1 DNA (BRL) and 2 μg glycogen (BMB) were prehybridized to a Cₒt value 4× greater than in the standard protocol. The prehybridized rat cDNA mixture was then mixed with the mouse template cDNA and hybridized essentially as in the standard protocol. After hybridization was stopped, the biotinylated material was washed 3×15 min in 0.1×SSC/0.1% SDS at one of the three wash temperatures 65° C., 55° C., or 50° C. (depending on the stringency desired). Finally, the selected cDNAs were eluted and eventually dU-cloned into the pAMP10 vector (BRL) by amplifying 30 cycles with 60° C. annealing using the cDNA-U-2 primer (5′ CUACUACUACUA GTCACGCAAGCTTCTCACAG 3′) (SEQ ID NO: 24).

Genotyping

DNA was extracted from rat tail biopsies obtained at 25-30 days of age. Genotyping for simple sequence repeats markers was carried out as previously described (Jacob et al., Nature Genetics 2:56-60 (1992)). Rat tail DNA was PCR amplified using IRD-700 tailed primers (LI-COR Biosciences, Lincoln, Nebr.). The PCR products were analyzed using a NEN Global IRZ DNA Analyzer System (Model 42005-2) using 6.5% gel matrix (LI-COR Biosciences, Lincoln, Nebr.).

DNA Sequence Analysis

Initial Sequence Analysis of BB DR. lyp.

Primer pairs were designed for amplification of the rIanS coding exons 2 and 3 (forward primer, 5′-GCTTGAGGAGGTCATCAGTTC-3′ (SEQ ID NO: 25) and reverse primer, 5′-CTCACGTCCCAGCCTCTAAC-3′ (SEQ ID NO: 26)). PCR reactions were 2 min at 95° C.; 10x: 30 s at 95° C., 30 s at 60° C., 30 s at 72° C.; 30×: 30 s at 95° C., 30 s at 60° C., 30 s plus 10 s/cycle at 72° C.; 7 min at 72° C. The PCR products were purified with Ultrafive-MC (Millipore, Bedford, Mass.). Purified PCR products (30-60 ng) were subjected to cycle sequence reactions using IRDye 800 terminators (LiCor) and Thermo Sequenase (USB). The reaction products were purified with a MultiScreen Filtration System (Millipore) using Sephadex TM G-50 Fine (Amersham Pharmacia Biotech, Alameda, Calif.) and analyzed using NEN Global IRz DNA Analyzer System sequencer (LI-COR Inc, Lincoln, Nebr.).

Sequencing of Additional Inbred Rat Strains

Primers were selected for PCR amplification of 500 bp (forward primer 5′-CCATGGCTTTGAGGAAC TATCC3′ (SEQ ID NO: 27) and reverse primer 5′-TGTGGGTGAAGAGGACAATCAT-3′ (SEQ ID NO: 28)) and 385 bp (forward primer 5′AAAGTGCCACAGGGAACAGC (SEQ ID NO: 29) and reverse primer 5′-GTGTGGGTCACAAACTCTTCCA-3′ (SEQ ID NO: 30)) fragments, encompassing the rIan5 deletion mutation. Amplified products were subject to standard fluorescent sequencing using an ABI3700 automated sequencer. Analysis was performed using Phred, Phrap, Consed, and Polyphred, to compare the sequences between BBDP and 38 other inbred rat strains.

In Silico Sequence Analysis

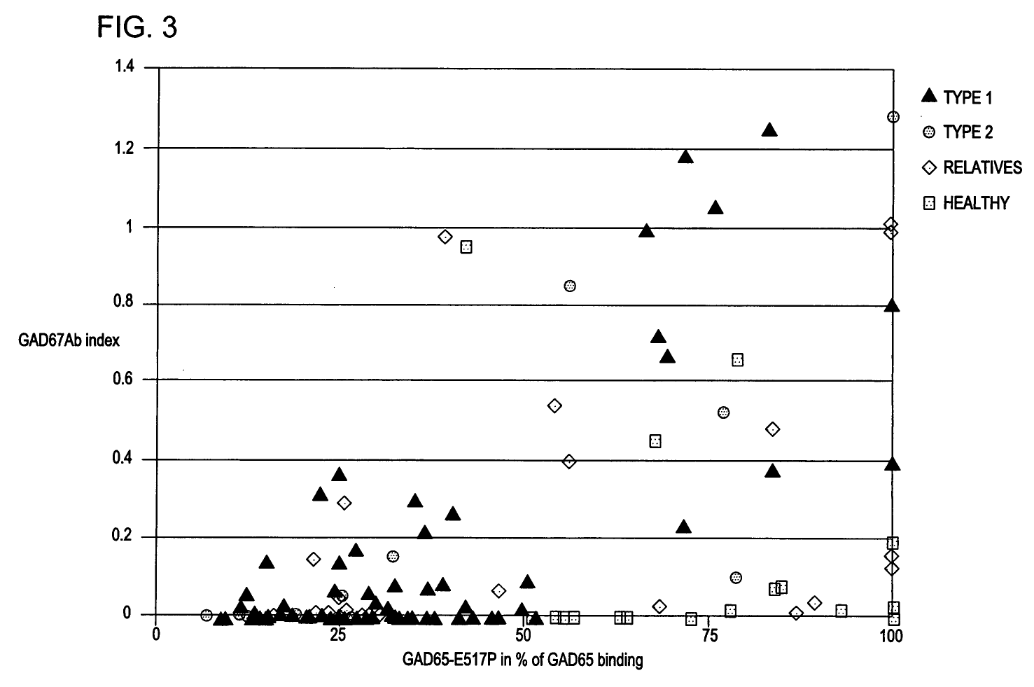

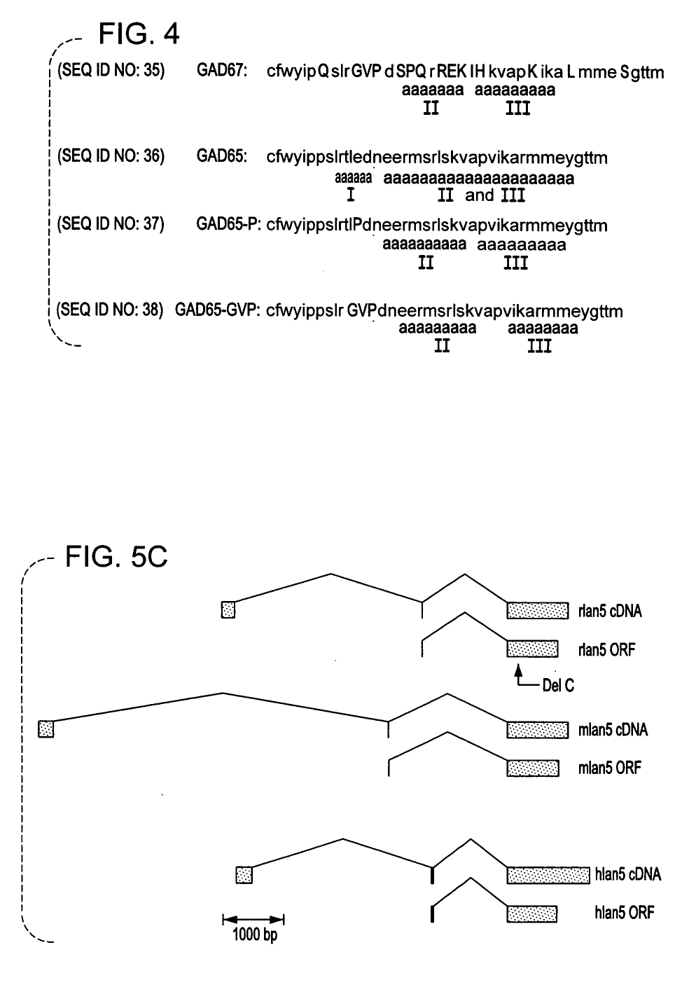

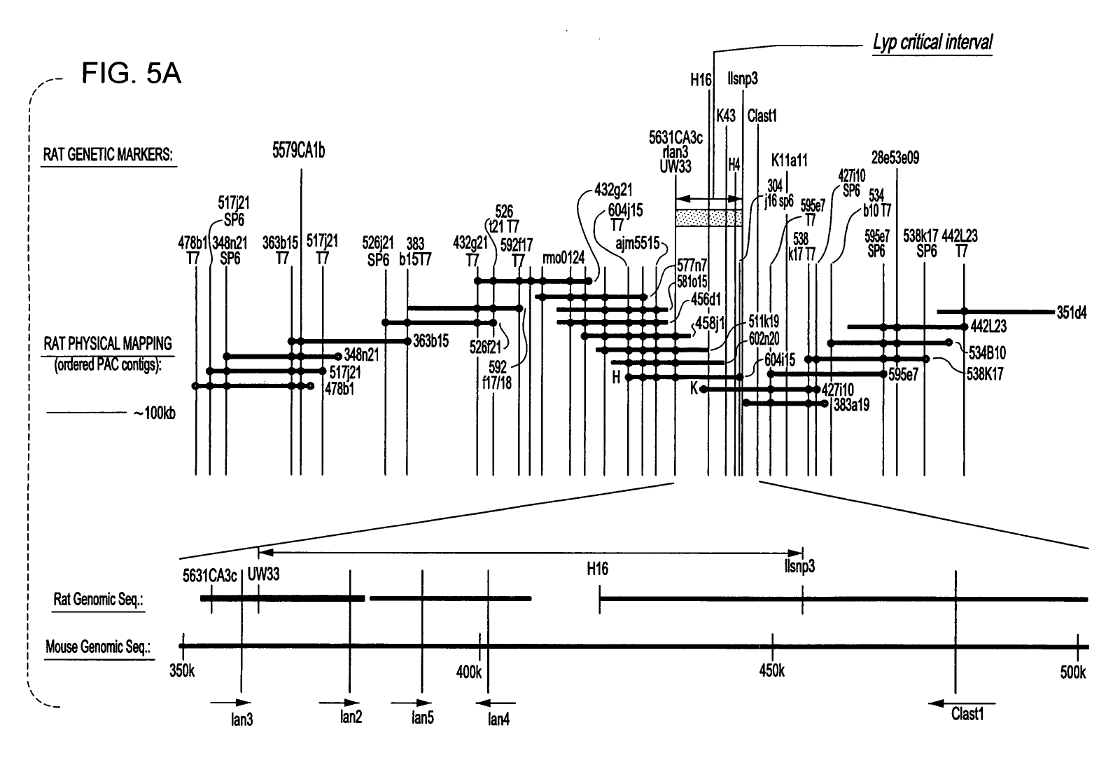

For the human, NCBI's genomic TBLASTN was used with the predicted protein product of hIan5 blasted against the GenBank human genome as of Dec. 24, 2001, setting the expectation parameter to 10. The E values of the resulting matches were bimodal, with the matches plotted in FIG. 5B ranging from e\hat{ }-167 to 4e\hat{ }-28 and the remaining spurious matches having E values >1.

For the mouse, TBLASTN was again used with the predicted protein product of mlan4 blasted against the GenBank mouse genome supercontig database (mgscv3) posted on Apr. 19, 2002, setting the expectation parameter to 10. Again the resulting E values were bimodal, with the FIG. 5B matches ranging from e\hat{ }-160 to 3e\hat{ }-8 and the remaining spurious match having an E value of 1.5. Also used were other NCBI Blast programs such as BlastN and Blast2 according to recommended settings, in order to identify homologous EST sequences, already-identified genes, and the alignments of one sequence within another (Altschul et al., J. Mol. Biol. 215:403-410 (1990); Altschul et al., Nucleic Acids Res. 25:3389-3402 (1997)).

RNA Isolation and Quantification of Ian5 mRNA Levels.

Organs were dissected from 49 day old congenic DR.lyp rats of each of three genotypes (wild-type or +/+, lypl+, and lyp/lyp) from the same litter and homogenized immediately. Poly A+ RNA was isolated using Qiagen RNeasy and Oligotex kits (Qiagen, Chatsworth, Calif.). Three μg of poly A+ RNA per well was electrophoresed through a 0.9% agarose gel containing MOPS buffer (40 mM 3N-morpholino-propane sulfonic acid [MOPS], 10 mM sodium acetate, 1 mM EDTA), and 2% formaldehyde. The gel was washed twice, 30 min in DEPC water, 35 min in 50 mM NaOH, 1.5 M NaCl, 30 min in 1 M Tris pH 8.0, 1.5 M NaCl, and 5 min in 10×SSC (1×SSC is 0.15 M NaCl, 15 mM Na citrate, pH 7.0). The RNA was transferred to a positively charged nylon membrane (Roche, Indianapolis, Ind.) by vacuum blotting and crosslinked to the membrane in a UV Stratalinker 1800 (Stratagene, La Jolla, Calif.). The membrane was stained with methlyene blue stain (0.03% methylene blue in 0.3 M sodium acetate, pH 5.2).

Blots were prehybridized for 1 hr in Church buffer (0.5 M Na phosphate buffer, pH 6.8, 1 mM EDTA, 7% SDS) at 65° C. ³²P-labeled probe was made by amplifying a 695 bp fragment by PCR using rIAN4-690f (5′-CTCCTGGTGGGTAAATCTGG-3′ (SEQ ID NO: 31)) forward primer and rIAN4-1384r (5′TCCTTCAGCTCCCTCTTCTG-3′ (SEQ ID NO: 32)) reverse primer (Invitrogen, Gaithersburg, Md.) in mix containing: 50 ng genomic DR.lyp DNA, 1×TAQ Polymerase Buffer, 250 μM each (dATP, dGTP, dTTP) and 50 μM dCTP, 0.5 μM each primer, 40 μCi ³²P dCTP (PerkinElmer Life, Sciences, Boston, Mass.), 0.5 U TAQ 2000 DNA Polymerase (Stratagene, La Jolla, Calif.). Probe was amplified at 95° C. for 3:00 min, then 35 cycles of (95° C. for 0:45, 60° C. for 0:45, 72° C. for 1:00 min).

The probe was purified through a G50 AutoSeq column (Amersham Pharmacia Biotech, Alameda, Calif.), denatured by heating at 96° C. for 7.5 min, iced, and added to the blot overnight at 65° C. The blot was rinsed twice with 2×SSC/0.1% SDS at room temperature, then washed with 2×SSC/0.1% SDS at 65° C. for 20 min, 0.2×SSC/0.1% SDS at 65° C. for 20 min, 0.1×SSC/0.1% SDS at 65° C. for 30 min. The blot was then placed on BioMax MS Film (Eastman Kodak, Rochester, N.Y.) and subsequently on a phosphor screen to be scanned by a STORM 840 phosphor imager and quantified with ImageQuant v1.2 software (Molecular Dynamics, Sunnyvale, Calif.). Blots were stripped by overnight wash in 0×SSC/0.1% SDS at 65° C. and reprobed with rat GAPDH (Accession no AB017801) cloned into pGEM3z. Using the same method described above, T7 (5′-TAATACGACTCACTATAGGG-3′ (SEQ ID NO: 33)) forward primer and T3 (5′-ATTAACCCTCACTAAAGGGA 3′ (SEQ ID NO: 34)) reverse primer were used to generate a 1420 bp fragment in mix containing: 1 ng pGEM3zrGAPDH, 1×Taq Polymerase Buffer, 250 μM each (dATP, dGTP, dTTP) and 50 μM dCTP, 0.5 μM each primer, 40 μCi ³²P dCTP (PerkinElmer Life Sciences, Boston, Mass.), 0.5 U TAQ2000 DNA Polymerase (Stratagene, La Jolla, Calif.). Probe was amplified at 95° C. for 3:00, then 35 cycles of (95° C. for 0:45, 50° C. for 0:45, 72° C. for 1:45).

Results

Comparative Genomics of the Iddml Genomic Interval

When the positional cloning of Iddml/lyp was begun, physical maps were not available for the rat. Therefore, a physical map of the syntenic region in the mouse was first constructed, with the expectation that the mouse ortholog of Iddml/lyp would lie in this interval. Comparative mapping determined that the Iddml/lyp region on rat chromosome 4 (between D4Mit6 and D4Mit24) is syntenic to the proximal end of mouse chromosome 6 (Mmu6). Gene order appears to be conserved between rat and mouse, over the region just proximal of the rat Iddml/lyp region (including the T cell receptor beta-chain genes) to 10-15 cM distal of the locus (including the immunoglobulin kappa chain complex, Igk) (data not shown). Because the mouse Iddml/lyp region was expected to contain the ortholog of the rat Iddml/lyp gene, it was determined that the genomic DNA of both the mouse and rat Iddml/lyp regions would be isolated, combining the information and reagents from both species in order to create a comprehensive map of the region. A mouse YAC contig was constructed spanning the approximately 2 Mbp interval of the mouse Iddml/lyp region, and gene fragments were isolated from that interval. The mouse gene fragments were used as probes to isolate the corresponding orthologous rat gene fragments by cross-species cDNA selection (Lovett et al., Biochem. Biophys. Res. Comm. 144:1069-1075 (1987)). Next, a rat YAC contig was constructed spanning the rat Iddml/lyp region by isolating those rat YAC clones that contained the rat gene fragments. STS content mapping and hybridization of gene fragments from one map to the other confirmed that the local gene order was the same in rat and mouse.

Characterization of the Rat, Human and Mouse lyp Regions

The initial mouse physical map was converted into a more useful higher-resolution form by isolating contigs of genomic BAC clones. Overlapping mouse BAC clones spanning over 800 kb of the mouse Iddml/lyp region were sequenced. STSs from these BAC clones were then used to refine the rat physical map by identifying corresponding rat PAC clones (rat contig shown in FIG. 5A), which were then sequenced. While generating the physical map of the Iddml/lyp regions in mouse and rat, recombinant animals were also generated to refine the position of Iddml/lyp on the rat genetic map (Table 3). BBDR (+/+) and DR.lyp (lyp/lyp) rats continued to be intercrossed; and DR.lyp (lyp/lyp) and F344 rats continued to be backcrossed and then intercrossed. These crosses provided over 300 additional animals in addition to the 870 animals already analyzed (Jacob et al., Nature Genetics 2:56-60 (1992)), totaling over 2400 meioses. Resulting recombinant animals identified the Iddml/lyp interval, flanked by an SSLP, UW33, on the proximal end and a SNP, IIsnp3, on the distal end (FIG. 5A). This region corresponds to approximately 100 kb on the mouse genome. With the assembly of a draft sequence of the mouse genome by the International Mouse Genome Sequencing Consortium (www.ensembl.org/Mus_musculus), the mouse sequence determined as described above was integrated to produce a contig including the entire mouse region orthologous to the rat lyp interval (FIG. 5B). The mouse genomic sequence was then aligned to the human syntenic region on chromosome 7q36.1 and evaluated the conserved genes annotated in both species.

TABLE 3
Recombinant genotypes specify the boundaries of the lyp critical interval in the rat.

| Cross | Rat | 5631CA3c | UW33 | | H16 | K43 | H4 | lyp | Ilsnp3 | C |
|---|---|---|---|---|---|---|---|---|---|---|
| BB-DP X BB-DR | 11CF2 19:2 | 3 | 3 | | 3 | 3 | 3 | + | nt | |
| | 2CF3 1:12 | 1 | 1 | | nt | nt | 1 | Lp | nt | X |
| BB-DP X F344 | 1FBF2 18:6 | 1 | 1 | | 1 | 1 | 1 | Lp | 111X33 | |
| | 2FBF2 11:5 | 1 | 1 | | 1 | 1 | 1 | Lp | 11111 | X |
| | 4FBF2 14:13 | 3 | 3 | | 3 | 3 | 3 | + | 33333 | |
| | 4FBF2 31:11 | 3 | 3 | X | 1 | 1 | 1 | Lp | 11111 | |
| The type of cross and the identification of the recombinant animals are listed to the left and the genotypes (or phenotypes in the case of lyp) at selected loci are shown. The key recombinant animals defining the lyp critical interval (indicated by dark vertical bars) are 4FBF2 31:11 on the left (proximal) side and 1 FBF2 18:16 on the right (distal) side. The “X” marks indicate the inferred locations of recombinant breakpoints, nt indicates genotypes not | | | | | | | | | | |
| # tested, Lp is lymphopenic (the cut off value is 15% representing the mean +4SD) and + indicates a normal (non-lyp) phenotype. SNP marker Ilsnp3 is not polymorphic in the BB-DP x BB-DR cross. | | | | | | | | | | |

A notable feature of this region is the presence of a family of at least ten putative GTP-binding protein genes found only in this region of the human and mouse genomes, the Immune Associated Nucleotide (IAN) gene family (Daheron et al., Nucleic Acids Res. 29:1308-1316 (2001); Krucken et al., J. Biol. Chem. 274:24383-24391 (1999); Poirier et al., J. Immunol. 163:4960-4969 (1999); Stamm et al., Gene 282:159-167 (2002); Cambot et al., Blood 99:3293-3301 (2002)). Interestingly, all Ian gene family members are located in a 300 kb interval within 7q36.1 and a more compact 120 kb region in the mouse. This may be a consequence of genomic rearrangement in the human Ian gene region relative to the mouse Ian gene region since the two species' evolutionary divergence, as the number of Ian genes differs in each (10 in human, 11 in mouse), and there are breaks in the gene order of the orthologs between the species (for example, hIanl2 has no ortholog in the mouse, and mIan3 is one of two orthologs of hIan7). The region in the mouse genome corresponding to the critical Iddml/lyp interval in rat was examined and it was found that three IAN family members lay within this critical region (FIG. 5A). While Ian2 was expressed in the spleen (Krucken et al., Biochem. Biophys. Res. Comm. 230:167-170 (1997)), Ian4 was only expressed at low levels but was not detected in any other lymphoid tissue (Daheron et al., Nucleic Acids Res. 29:1308-1316 (2001)). The third gene, designated Ian5 has not previously been reported in mouse or rat. Rat Ian3 is differentially expressed in thymus and spleen when comparing tissue from DR.lyp +/+ and lyp/lyp rats, although the rat Ian3 gene is outside the defined region.

Identification of rIan5 that Contains a One bp Deletion Unique to the DR.lyp Rat

The intron/exon structure of the rIan5 gene is shown in FIG. 5C in comparison with its mouse and human orthologs, mIan5 and IAN4L1 (hIan5). The overall genomic structure is similar to that reported in this family of genes (Stamm et al., Gene 282:159-167 (2002)). As with hIan5, rIan5 has at least three exons. The first and second exons are short, 220 bp and 49 bp, respectively, while the last exon is 1047 bp. There is a 3895 bp intron between exons 1 and 2, and a 1457 bp intron between exons 2 and 3. Exon 2 contains the putative start site for the major ORF spanning exons 2 and 3, as reported for mlan4 (Daheron et al., Nucleic Acids Res. 29:1308-1316 (2001)). Exons 1 and 2 contain an additional 61 as ORF starting at position 78; this overlaps the major ORF and has no significant amino acid sequence similarity with the small 5′ ORF in mIan4.

It was established that rIan5 is expressed in rat spleen, thymus, and lymph nodes, making it a strong positional candidate for Iddml/lyp in the BB rat. To identify potential functional variants in this gene in the DR.lyp rats, primers spanning the putative coding sequence were used to amplify and sequence the gene from both BBDR wild-type and DR.lyp (lyp/lyp) thymic cDNA. The cDNA sequence was confirmed by sequencing of BBDP/WorAp and BBDR as well as F344 rat genomic DNA (FIG. 6). The sequence analysis showed that both the DR.lyp and BBDP/WorAp strains lack one C nucleotide at bp position 478 of rIan5, causing a frameshift mutation in the presumed—ORF (exon 3) and leading to a truncated mutant predicted protein product (FIG. 7). The frameshift deletion in the lyp/lyp rIan5 changes the predicted downstream amino acids to include 19 amino acids (boxed) before the premature STOP codon (FIG. 7). It was confirmed that this nucleotide deletion was present in our lymphopenic congenic F344.1yp inbred rat line, as well as in outbred BBDP (diabetes prone) rats from Ottawa (Table 4). As expected, the non-lymphopenic, diabetes resistant outbred BBDR rat from Ottawa did not contain this deletion. In order to determine whether the frameshift deletion was a common polymorphism among rat strains, or mutation unique to strains with lymphopenia, approximately 500 bp of rIan5, encompassing the deletion, were resequenced in 38 inbred rat strains (Table 4). The different strains have been characterized with genetic markers spanning the genome and were selected to represent inbred lines or strains of rats with maximum genetic diversity (Steen et al., Genome Res. 9:AP1-8 (1999)); only the DR.lyp and BBDP/WorAp strains have lymphopenia and type 1 diabetes. The frameshift mutation was found only in the strains with lymphopenia (DR.lyp and BB-DP). Three other sequence variants were found among the 38 strains and can be summarized as three distinct haplotypes (Table 4). The most common haplotype was found in 26 of the 38 strains; the frameshift mutation occurs on this haplotype. While the normal rIan5 sequence predicts a protein of 35 kD, the deletion mutant would represent a dramatically truncated protein of 11 kD.

TABLE 4
Sequence analysis of Ian5 in different inbred strains of rats.

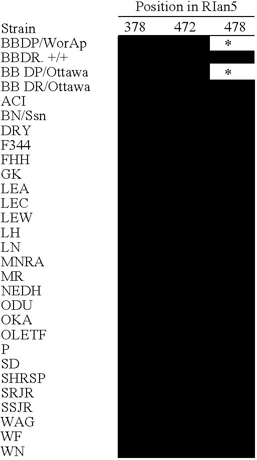

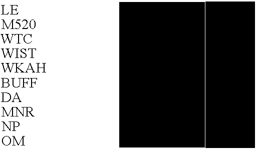

Inbred strain designations and descriptions can be found on RGD (http://rgd/mcw.edu). Genotypes are listed at the specified positions in rIan5. Lighter shading represents the most common allele. Darker shading represents the less common SNP. The deletion found in diabetic prone BB rat is designated by *.

rIan5_{del} Expression Reduced in Hematopoetic Cells

Northern blot analysis of polyA+ RNA prepared from DR.+/+ and lyp/lyp rat tissues showed that the rIan5 transcript of 1.4-kb was expressed in the thymus and spleen but not in kidney (FIG. 8A). The transcript levels were markedly reduced in lyp/lyp as compared to +/+tissue. Ian5 mRNA levels in thymus of lyp/lyp animals were reduced to 45% of wild type levels. The level was even lower in spleen of lyp/lyp animals (only 6% of wild type levels) although this may reflect the absence of T cells resulting from the lymphopenia phenotype. Expression levels in the kidney were extremely low (3% of wild type thymus) and differences between phenotypes could not be reliably observed. To prove that the decreased expression levels were a direct result of the frameshift mutation, and not a secondary consequence of the absence of T cells due to lymphopenia, expression levels in lyp/+heterozygotes which show no lymphopenia were examined. Heterozygotes showed intermediate levels of expression (FIG. 8B). These data support the notion that the frameshift mutation in the rIanS gene causes a marked reduction in the mRNA in hematopoietic tissues previously established to be affected by T-cell lymphopenia and results in the lymphopenia and diabetes in the diabetic prone BB rat. The reduced levels of rIan5 transcription found in lyp/+heterozygotes cannot be explained on the basis of T cell numbers since both wildtype and heteozygotes have normal numbers of thymocytes and peripheral T cells but may rather be due to the possibility that the mutated rIan5 transcripts are unstable.

The previous examples are provided to illustrate but not to limit the scope of the claimed invention. Other variants of the inventions will be readily apparent to those of ordinary skill in the art and encompassed by the appended claims. All publications, patents, patent applications and other references cited herein are hereby incorporated by reference.

What is claimed is:

1. A GAD65 polypeptide comprising an E517P mutation, said polypeptide characterized by decreased specific binding to an antibody selected from the group consisting of GAD6, MICA-1, MICA-3, MICA-4, and MICA-6, wherein the decreased binding is relative to a corresponding GAD65 polypeptide not having the E517P mutation.

2. The mutant GAD65 polypeptide of claim 1, further characterized by decreased specific binding to MICA-2 antibody, wherein the decreased binding is relative to a corresponding GAD65 polypeptide not having the E517P mutation

3. The mutant GAD65 polypeptide of claim 1 which is a full-length GAD65 polypeptide.

4. The mutant GAD65 polypeptide of claim 1, which comprises a sequence at position 515-525 that is selected from group consisting of SEQ ID NO: 15 and SEQ ID NO:16.

5. A method for detecting the presence of or risk of type 1 diabetes in a subject, the method comprising:
1) isolating from the subject a first serum sample and a second serum sample;
2) contacting the first serum sample with a mutant GAD65 polypeptide according to claim 1;
3) contacting the second serum sample with a control GAD65 polypeptide that is immunologically cross-reactive with C-terminal conformational epitopes of wild-type GAD65, said control GAD65 polypeptide not having the E517P mutation and having substantially the same GAD6-, MICA-1-, MICA-3-, MICA-4-, or MICA-6-specific binding activity as the corresponding GAD65 polypeptide;
4) determining the degree of GAD65 autoantibody binding activity in the first and second serum samples; and
5) comparing the degree of GAD65 autoantibody binding activity in the first and second serum samples to detect the presence of or risk of type 1 diabetes in the subject.

6. The method of claim 5, wherein the control GAD65 polypeptide is the corresponding GAD65 polypeptide not having the E517P mutation.

7. The method of claim 5, wherein the control GAD65 polypeptide is wild-type GAD65.

8. The method of claim 5, wherein the control GAD65 polypeptide has the amino acid sequence of SEQ ID NO: 12 at position 515-525.

9. The method of claim 5, wherein the mutant GAD65 polypeptide is full-length.

10. The method of claim 5, wherein the mutant GAD65 polypeptide consists of the GAD65 wild-type amino acid sequence at positions other than position 517.

11. An isolated nucleic acid selected from the group consisting of:
(a) nucleic acids which encode the rat Ian5(+) polypeptide of SEQ ID NO: 3;
(b) nucleic acids which encode the rat Ian5(lyp) polypeptide of SEQ ID NO: 4; and
(c) full length complements of the nucleic acids of (a) or (b).

12. The isolated nucleic acid of claim 11, which comprises a nucleotide sequence selected from the group consisting of (a) SEQ ID NO: 1 and (b) nucleotides 1-312 of SEQ ID NO: 2.

13. An isolated polypeptide comprising the amino acid sequence of SEQ ID NO: 3.

14. An isolated polypeptide consisting essentially of amino acids 1-84 of SEQ ID NO: 4.

15. The isolated polypeptide of claim 14, further consisting of 1-40 random amino acids adjacent and carboxy terminal to amino acid 84.

16. The isolated polypeptide of claim 15, which is the polypeptide having the amino acid sequence of SEQ ID NO: 4.

17. An isolated polypeptide consisting essentially of amino acids 1-85 of SEQ ID NO: 6.

18. The isolated polypeptide of claim 17, further consisting of 1-40 random amino acids adjacent and carboxy terminal to amino acid 85.

19. An antibody that specifically binds to rat Ian5(+) polypeptide, wherein said antibody is not immunologically cross-reactive with human Ian5 or mouse Ian5 polypeptide.

20. The antibody of claim 19, which is a monoclonal antibody, a polyclonal antibody, a single chain antibody, a heavy chain antibody, an F(ab′)₂, F(ab′), or Fᵥ fragment.

21. An antibody that specifically binds to rat Ian5(lyp) polypeptide, wherein said antibody is not immunologically cross-reactive with rat Ian5(+), human Ian5, or mouse Ian5 polypeptide.

22. The antibody of claim 19, which is a monoclonal antibody, a polyclonal antibody, a single chain antibody, a heavy chain antibody, an F(ab′)₂, F(ab′), or Fᵥ fragment.

23. An expression construct comprising the following elements linked in operable combination:
a transcriptional promoter;
a nucleic acid according to claim 11, and
a transcriptional terminator.

24. The expression construct of claim 23, wherein the nucleic acid comprises a nucleotide sequence selected from the group consisting of SEQ ID NO: 1 and nucleotides 1-312 of SEQ ID NO: 2.

25. A prokaryotic or eukaryotic cell transformed or transfected with the expression construct according to claim 23.

26. The prokaryotic or eukaryotic cell of claim 25, which is selected from the group consisting of a bacterial cell, a yeast cell, and a mammalian cell.

27. A vector comprising the expression construct according to claim 23.

28. An isolated host cell comprising the vector of claim 27.

29. A method for producing an Ian5 polypeptide, the method comprising:
growing cells transformed or transfected with the vector of claim 27; and
isolating the Ian5 polypeptide from the cells.

30. The method of claim 29, wherein the cells are selected from the group consisting of bacterial cells, yeast cells, and mammalian cells.

31. An in vitro method of identifying agonists or antagonists of an Ian5 pathway to identify candidates for type 1 diabetes drug development, the method comprising:
administering a candidate compound to a first cell that expresses the Ian5 polypeptide;
administering the candidate compound to a second cell that does not express the polypeptide; and
determining whether the candidate compound produces a physiological change in the first cell relative to the second cell.

32. The method of claim 31, wherein the first and second cells are mammalian cells.

33. The method of claim 32, wherein the mammalian cells are mammalian hematopoietic cells.

34. The method of claim 32, wherein the Ian5 polypeptide is selected from the group consisting of rat Ian5(+), rat Ian(lyp), human Ian5, and mouse lan5.

35. The method of claim 31, wherein the candidate compound stimulates or inhibits cell proliferation.

36. A method for developing gene therapy for type 1 diabetes, the method comprising:
(1) administering a nucleic acid comprising an Ian5 polynucleotide to a non-human mammal having a frameshift mutation in the Ian5 gene locus, wherein said frameshift mutation results in a truncated mutant Ian5 polypeptide consisting essentially of amino acids corresponding to amino acids 1-84 of SEQ ID NO: 4; and wherein said non-human animal exhibits at least one clinical symptom of type 1 diabetes; and
(2) determining whether the nucleic acid encoding the Ian5 polynucleotide produces an amelioration of at least one clinical symptom of diabetes.

37. The method of claim 36, wherein the nucleic acid comprising the Ian5 polynucleotide is selected from the group consisting of:
(a) a vector comprising an Ian5 polynucleotide that encodes a wild-type Ian5 polypeptide, said wild-type Ian5-encoding polynucleotide flanked by regions that promote intrachromosomal homologous recombination;
(b) a vector comprising, linked in operative combination, a transcription promoter, the wild-type Ian5-encoding polynucleotide as in (a), and a transcription terminator;
(c) an antisense Ian5 polynucleotide hybridizable within a cell to a polynucleotide encoding the truncated mutant Ian5 polypeptide; and
(d) a vector comprising, linked in operative combination, a transcription promoter, the antisense Ian5 polynucleotide as in (c), and a transcription terminator.

38. The method of claim 36, wherein the non-human mammal is a genetically modified mammal having an exogenous mutant Ian5 gene.

39. The method of claim 36, wherein the non-human mammal having the frameshift mutation is a DR.lyp/lyp rat.

40. A method for developing gene therapy for type 1 diabetes, the method comprising:
(1) administering a vector comprising an Ian5 polynucleotide to a non-human mammal having a knockout mutation in the Ian5 gene locus, wherein said non-human animal exhibits at least one clinical symptom of type 1 diabetes; and
(2) determining whether the vector produces an amelioration of at least one clinical symptom of diabetes.

41 The method of claim 40, wherein the vector is selected from the group consisting of:
(a) a vector comprising an Ian5 polynucleotide that encodes a wild-type Ian5 polypeptide, said wild-type Ian5-encoding polynucleotide flanked by regions that promote intrachromosomal homologous recombination; and
(b) a vector comprising, linked in operative combination, a transcription promoter, the wild-type Ian5-encoding polynucleotide as in (a), and a transcription terminator.

42. The method of claim 40, wherein the non-human animal having the Ian5 knockout mutation is a transgenic knockout mouse.

43. A method for detecting in a subject the presence of or risk of developing type 1 diabetes, the method comprising:
detecting the presence of a mutation at one or more nucleotide positions in the Ian5 gene in a sample from the subject; and therefrom identifying the presence or risk of developing type 1 diabetes.

44. The method of claim 43, wherein the mutation is a frameshift mutation resulting is a truncated mutant Ian5 polypeptide.

45. The method of claim 44, wherein the mutation is a mutation in codon 85 of the human Ian5 coding sequence.

46. The method according to claims 43, wherein the presence of the mutation is detected by a technique that is selected from the group consisting of direct sequencing, hybridization with oligonucleotide probes, a ligation reaction, a polymerase chain reaction, and single nucleotide primer-guided extension assays.

47. A method for identifying a genetic mutation that correlates with type 1 diabetes, the method comprising:
(a) determining the sequence of the Ian5 gene from a plurality of humans known to have diabetes;
(b) comparing the sequence to the wild-type human Ian5 gene sequence; and
(c) identifying mutations in the human Ian5 genes that correlate with the presence of type 1 diabetes.

48. A method for detecting in a human subject the presence of or the risk of developing type 1 diabetes, the method comprising:
(a) obtaining from the subject a biological sample containing or derived from lymphocytes;
(b) obtaining a control sample containing or derived from lymphocytes;
(c) determining the level of Ian5 gene expression in the subject sample and the control sample; and
(d) comparing the level of Ian5 gene expression in the subject sample and the control sample to detect the presence of or the risk of developing type 1 diabetes.

49. The method of claim 48, wherein the level of Ian5 gene expression is determined with a nucleic acid probe.

50. The method of claim 49, wherein the level of Ian5 gene expression is determined with an anti-Ian5 antibody.
